(19) United States
(12) Patent Application Publication
(10) Pub. No.: US 2025/0079008 A1
(43) Pub. Date: Mar. 6, 2025

(54) DEPLOYMENT AND USE OF A CONTINUOUS ANALYTE MONITORING SYSTEM FOR IMPROVED PATIENT TREATMENT

(71) Applicant: Abbott Diabetes Care Inc., Alameda, CA (US)

(72) Inventors: Paul R. E. JARVIS, Pudsey (GB); Junli OU, Pleasanton, CA (US); Ting CHEN, Alameda, CA (US); Lorelie H. VILLARETE, Alameda, CA (US); James MCCARTER, St. Louis, MO (US); Matthew BATES, New Hope, PA (US); Naveen THURAMALLA, Dublin, CA (US); Philip B. ADAMSON, Austin, TX (US)

(21) Appl. No.: 18/822,170

(22) Filed: Aug. 31, 2024

Related U.S. Application Data
(60) Provisional application No. 63/580,305, filed on Sep. 1, 2023.

(51) Int. Cl. G16H 50/20; A61B 5/00; A61B 5/145; G16H 10/60; G16H 20/10

(52) U.S. Cl. CPC G16H 50/20; A61B 5/14532; A61B 5/14546; A61B 5/4839; A61B 5/7264; A61B 5/7275; A61B 5/746; G16H 10/60; G16H 20/10; A61B 2560/045

(57) ABSTRACT

Disclosed herein are system, method, and computer program product embodiments for improving detection and treatment of patient conditions based on continuous analyte data. The disclosed techniques utilize analyte data, such as lactate, glucose, and creatinine, provided from a continuous analyte sensor to predict patient outcomes. The prediction may also take into account other medical information associated with the patient, such as patient vital signs and medical history. The disclosed system allows for early and non-invasive prediction of patient outcomes in various settings including a hospital setting, a home setting, disease (e.g., heart failure, sepsis) detection, and high risk surgery monitoring. The disclosed system also is configured to monitor patient conditions and generating alerts and/or notifications based on the predicted patient outcomes to provide preemptive treatment of patient conditions.

### FIELD OF THE INVENTION

This disclosure generally relates to continuous analyte sensors, including in vivo analyte sensors for sensing and monitoring analytes in a bodily fluid, and the use of such sensors for predicting patient outcomes and improving alerts and notifications. Specifically, embodiments described herein relate to a continuous analyte monitoring system in communication with one or more analyte sensors and utilizing the continuous analyte data in combination with other patient data as inputs to a patient prediction model. The prediction model is configured to operate in various settings, such as a hospital setting, home or other non-hospital setting, and/or in combination with various disease implementations such as heart failure and sepsis.

### BACKGROUND

Current methodologies for treating and monitoring patients can present challenges because they rely on invasive procedures, such as serial blood draws and surgical devices, and because they are reactive in nature. That is, current methodologies usually provide treatment just before or as a patient's medical condition deteriorates or have large time intervals between data points. This is because current methodologies do not constantly monitor patient conditions and use the monitored information to predict potential patient outcomes, which could lead to earlier identification of deterioration and earlier administration of preventative and/or curative treatment.

### SUMMARY

Accordingly, what is needed is a system that continuously monitors a patient's analyte levels, generates predicted patient outcomes based on the continuously monitored data, and facilitates earlier treatment decisions for the patient based on the predicted patient outcome.

The detection and monitoring of various analytes within an individual can be vital for the condition of their health and well-being. Deviation from normal analyte levels can often be indicative of an underlying physiological condition, such as a metabolic condition or illness, or exposure to particular environmental factors or stimuli. Glucose levels, for example, can be particularly important to detect and monitor in diabetic individuals.

Lactate is an analyte in which in vivo levels may vary in response to numerous environmental or physiological factors including, for example, eating, physiological stress, exercise, sepsis or septic shock, infection, hypoxia, hypoperfusion, increased metabolic demand, and the like. In the case of conditions such as sepsis or septic shock, periodic laboratory measurements of lactate levels may be insufficient to determine whether these conditions are increasing or decreasing in severity, and/or if the patient is responding to treatment. Other lactate-altering conditions may be episodic in nature, in which case lactate levels may fluctuate very rapidly and irregularly. Other use-cases in which continuous analyte data, including continuous lactate data, may be used for predicting patient outcomes include patients in a hospital setting, patients in a home or other non-hospital setting, disease prediction and detection such as sepsis and cardiac heart failure, and patients that have undergone certain kinds of surgical procedures. Continuous analyte data includes an analyte level, e.g., concentration, at a specific time and analyte levels measured over a period of time. Conventional laboratory measurements may be ill-suited to determine lactate levels in such instances. Namely, lactate levels may have changed several times between successive measurements, and an abnormal lactate level may be completely missed in such instances, thereby leading to potentially incorrect or delayed diagnoses. In the case of rapidly fluctuating lactate levels, it can be desirable to measure an individual's lactate levels continuously, such as through using an implanted in vivo lactate sensor. Even if a lactate spike is observed when measuring lactate levels with periodic laboratory measurements or point-of-care tests, there often is no possibility of taking proactive actions to alleviate or remediate a particular condition leading to the increased lactate levels. This can have significant consequences for a patient's health and well-being in some cases.

At least a portion of an in vivo analyte sensor is positioned in contact with a bodily fluid of the patient under the skin so as to provide continuous analyte data to connected devices. Continuous lactate monitoring can also be advantageous in individuals with chronic, fast or slow changing lactate levels as well. For example, continuous lactate monitoring can avoid the pain and expense associated with conducting multiple blood draws for assaying lactate levels.

According to some embodiments, a continuous analyte monitoring (CAM) system is configured with one or more analyte sensors in communication with one or more reader devices and user devices, which are configured to receive the continuously monitored analyte data. The CAM system may include a prediction model which predicts patient outcomes for different settings-such as a hospital setting, a home setting, disease detection, and high-risk surgery monitoring-based on at least the continuous analyte data. In some embodiments, the analyte may be any combination of lactate and another analyte, such as glucose or creatinine. In some embodiments, the prediction model also bases the predicted patient outcomes on other medical information associated with the patient such as the patient's medical history, prior and current treatments, prior and current vital signs, trend information, and health care provider (HCP) notes and preferences. The CAM system may be further configured to provide alerts, notifications, and/or recommendations for treatment based on the predicted patient outcome, and in some embodiments, HCP preferences, and communication settings. Depending on the type of setting, such as a hospital, the alerts, notifications, and/or recommendations may be transmitted to different devices.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1 is a diagram illustrating an operating environment of an example continuous analyte monitoring system for use with the techniques described herein.

FIG. 2 is a block diagram illustrating an example data receiving device for communicating with a sensor according to some embodiments.

FIG. 3 is a block diagram depicting an example analyte sensor device for communicating with a continuous analyte monitoring system according to some embodiments.

FIG. 4A is a diagram illustrating various operating environments of a continuous analyte monitoring system with a prediction model according to some embodiments.

FIG. 4B is a diagram illustrating various operating environments of a continuous analyte monitoring system with a distributed prediction model according to some embodiments.

FIG. 5 is a flowchart illustrating a method for utilizing a continuous analyte monitoring system within a hospital environment, according to some embodiments.

FIG. 6 is a flowchart illustrating a method for utilizing a continuous analyte monitoring system within a home environment, according to some embodiments.

FIG. 7 is a flowchart illustrating a method for utilizing a continuous analyte monitoring system in the detection of heart failure conditions, according to some embodiments.

FIG. 8 is a flowchart illustrating a method for utilizing a continuous analyte monitoring system in the detection of sepsis conditions, according to some embodiments.

FIG. 9 is a flowchart illustrating a method for utilizing a continuous analyte monitoring system during high-risk surgery procedures, according to some embodiments.

FIG. 10 is a chart illustrating an early warning system connection between trends, amount of measured continuous analyte data, and recommendations, according to some embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for an improvement patient treatment system through the use of a continuous analyte monitoring system and a prediction model for generating potential patient outcomes based on the continuously monitored analyte levels. The potential patient outcomes may be used to dynamically adjust alerts and notifications and direct patient treatment decisions.

During acute illness or injury, levels of the hormone epinephrine are raised. This leads to the conversion of glucose into lactate, increasing circulating levels. In addition, poor tissue oxygenation, primarily due to a drop in blood pressure, may lead to anaerobic respiration which further contributes to rising lactate levels. Embodiments of the present disclosure utilize continuous lactate (and in some additional embodiments, analyte) monitoring for measuring lactate levels. A prediction model may be updated based on the monitored lactate information which improves the accuracy of the prediction of patient outcomes as well as the capability to dynamically adjust patient treatment. Rises in lactate levels are associated with worse clinical outcomes and could be used to direct treatment decisions. Continuous lactate monitoring could be used to monitor patients, screening for signs of deterioration. This would reduce the blind spot between nursing observations, potentially allowing for earlier identification of patient decline. This could lead to a reduction in patient mortality, reduction in intensive care unit (ICU) admission, and shorten both ICU and overall inpatient length of stay.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

As used herein and in the appended claims, the singular forms “a,” “an,” and “the” include plural referents unless the context clearly dictates otherwise. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “continuous” as it relates to a continuous analyte sensor refers to a sensor that is configured to take one or more measurements of the analyte over a period of time. A continuous sensor may take sequential measurements according to its sampling frequency. For example, one or more measurements may be taken about every 1 ms, about every 10 ms, about every 100 ms, about every 1 s, about every 10 seconds, about every 30 seconds, about every minute, about every 5 minutes, about every 10 minutes, about every 30 minutes, or about every hour. The measurements may be taken continuously e.g. over a contiguous time period of at least 1 hour, 6 hours, 12 hours, 1 day, 2 days, 3 days, 4 days, 5 days, 6 days, 1 week, 2 weeks, 3 weeks, 1 month or longer. A continuous analyte sensor is typically continuously in contact with a sample, such as a biofluid. For example, a continuous analyte sensor may comprise an implantable portion or member which in use is in continuous contact with a biofluid such as dermal fluid or interstitial fluid, such that measurements can be taken continuously or periodically according to the sampling frequency of the sensor over the continuous time period.

As used herein, the term “measure” and variations thereof can encompass the meaning of a respective term, such as “determine,” “calculate,” and variations thereof.

As used herein, a “sensor” is a device configured to detect the presence and/or measure the level of an analyte in a sample, including a continuous analyte sensor which is configured to detect analytes in the sample in a continuous manner.

The term “patient” refers to a living animal, and thus encompasses a living mammal and a living human, for example. The term “user” can be used herein as a term that encompasses the term “patient.”

Generally, embodiments of the present disclosure include systems, devices, and methods for the use of in vivo analyte monitoring systems for multiple different use cases.

Furthermore, many embodiments include in vivo analyte sensors structurally configured so that at least a portion of the sensor is, or can be, positioned in the body of a patient to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “analyte sensor” or “sensor” can refer to any device capable of receiving sensor information from a patient, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information.

Before describing aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. Continuous analyte monitoring (CAM) systems, for example, can transmit analyte data from a sensor control device to a reader device continuously without prompting, e.g., automatically according to a schedule such as updates every 1 minute, 5 minutes, or other interval, when within communication range, such as a Bluetooth connection range. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the patient, which can be analyzed to determine the patient's blood sugar level.

In vivo monitoring systems can include a sensor that, while positioned in vivo, makes contact with the bodily fluid of the patient and senses the analyte levels contained therein. The sensor can be part of the sensor control device that resides on the body of the patient and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the patient. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary Continuous Analyte Monitoring System

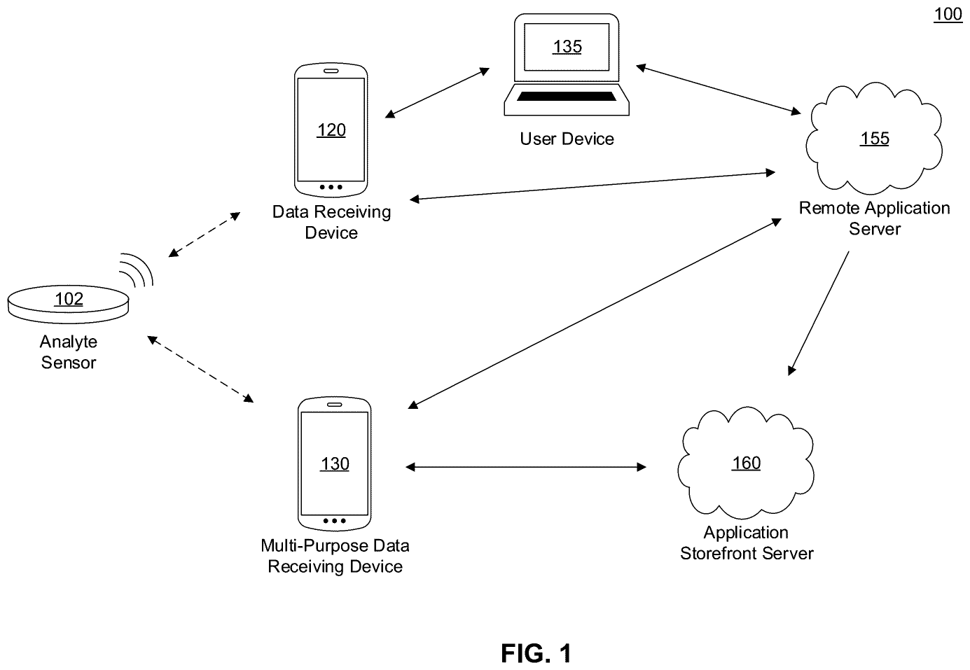

FIG. 1 illustrates an operating environment of a continuous analyte monitoring system 100 capable of embodying the techniques described herein. The continuous analyte monitoring system 100 can include a system of components designed to provide monitoring of parameters, such as analyte levels, of a human or animal body or can provide for other operations based on the configurations of the various components. As embodied herein, the system can include a low-power analyte sensor control device 102, or simply “sensor” worn by the patient or attached to the body for which information is being collected. As embodied herein, the analyte sensor control device 102 can be a sealed, disposable device with a predetermined active use lifetime (e.g., 1 day, 14 days, 30 days, etc.). Sensors 110 can be applied to the skin of the patient body and remain adhered over the duration of the sensor lifetime or can be designed to be selectively removed and remain functional when reapplied. The low-power continuous analyte monitoring system 100 can further include a data reading device 120, multi-purpose data receiving device 130, and user device 135 configured as described herein to facilitate retrieval, delivery, and display of data, including analyte data, from the analyte sensor control device 102.

One or more components of analyte system 100 may be physically located in one or more locations, such as the same location or distributed across different locations such as at a nurse's station or other center. For example, analyte sensor control device 102 is physically worn by the patient who may be located at a home location or in a hospital setting; data receiving device 120 may be located in physical proximity to the analyte sensor control device 102 (e.g., such as in the same room as the analyte sensor control device 102) or in a separate physical location but in network communication with the analyte sensor control device 102. Similarly, other components of analyte system 100 may be located in physical proximity to the analyte sensor control device 102 or in a separate physical location.

As embodied herein, the continuous analyte monitoring system 100 can include a software or firmware library or application provided, for example via a remote application server 150 or application storefront server 160, to a third-party and incorporated into a multi-purpose data receiving device 130 such as a mobile phone, tablet, personal computing device, or other similar computing device capable of communicating with the analyte sensor control device 102 over a communication link. Multi-purpose hardware for multi-purpose data receiving device 130 can further include embedded devices, including, but not limited to insulin pumps or insulin pens, having an embedded library configured to communicate with the analyte sensor control device 102. For example, multi-purpose data receiving device 130 may be a mobile phone or tablet configured to communicate with analyte sensor control device 102 via an installed application or software program. When the application or software program are uninstalled, multi-purpose data receiving device 130 may be unable to communicate with analyte sensor control device 102. The installed application may configure multi-purpose data receiving device 130 so that it can process analyte data. Some examples of processing data include displaying the analyte data, analyzing the analyte data, and generating visualizations of the analyte data.

As embodied herein, the continuous analyte monitoring system 100 can further include a data receiving device 120 capable of communicating with analyte sensor control device 102 over a communication link and multi-purpose data receiving device 130. In contrast to multi-purpose data receiving device 130, data receiving device 120 may be a device dedicated (e.g., without having to install any additional application or software) to communicating with and processing data from analyte sensor control device 102. In other embodiments, data receiving device 120 may be configured to communicate only with analyte sensor control device 102 and only process and display data received from and associated with analyte sensor control device 102.

As embodied herein, the continuous analyte monitoring system 100 can further include user device 135, which may be configured to be in communication with data receiving device 120, multi-purpose data receiving device 130, and/or a remote application server 155. In some embodiments, user device 135 may be implemented as a computer with a fixed location while data receiving device 120 and multi-purpose data receiving device 130 are implemented as mobile computers capable of being transported (e.g., with the patient, with a hospital bed). User device 135 may receive analyte data from data receiving device 120 and multi-purpose data receiving device 130 and provide additional output, input, and processing capabilities for interacting with the analyte data. For example, user device 135 may be implemented as a laptop or a personal computer that includes a keyboard, mouse, and/or a larger screen for displaying the analyte data. One or more components of continuous analyte monitoring system 100 may be deployed within different physical settings, such as a hospital or home, or different physical use cases, such as patients identified with risk of certain diseases, such as sepsis or congestive heart failure, or have undergone high-risk surgery. Implementation of the components (e.g., data receiving device 120, multi-purpose data receiving device 130, user device 135) may be based on the setting in which continuous analyte monitoring system 100 is deployed. For example, in a hospital setting, one or more data receiving device 120 or multi-purpose data receiving device 130 may be implemented as a patient bedside monitoring device configured to receive and display analyte data provided by analyte sensor control device 102 and user device 135 may be implemented as a computing device located in a different physical location within the hospital, such as a nurse's station. Remote application server 155 may be configured with modules for storing and configuring electronic medical records (EMR) for patients in one or more hospitals. Remote application server 155 is configured to receive analyte data from user device 135 and/or data receiving device 120 and further configured to update EMR profiles associated with the received analyte data.

Communication between components may also be based on the type of components. For example, the frequency of communicating analyte data between analyte sensor control device 102 and data receiving device 120 and/or user device 135 may be different from the frequency that the analyte data is communication to the remote application server. For example, data receiving device 120 and/or user device 135, when implemented in a hospital setting, may be utilized to display patient conditions in real-time; accordingly, the frequency of communication between analyte sensor control device 102 and data receiving device 120 and/or user device 135 may be configured to a high frequency to ensure that analyte data displayed by data receiving device 120 and/or user device 135 is accurate. On the other hand, the frequency of communication between data receiving device 120 and/or user device 135 and remote application server 155 may be set to a lower frequency (i.e., data is transmitted in batches) for storage at the remote application server 155.

Parameters of the communications between components may be dynamically updated based on any number of conditions including the component receiving the analyte data, the type of analyte being monitored, risk factors associated with the patient, time of day, current location of the patient, and a current condition of the patient. For example, there may be configurable threshold settings associated with these conditions that may trigger changes to the communication frequency. As one non-limiting example, a negative change in a patient's condition based on a certain threshold may trigger an increase in communication frequency. As another example, communications during evening hours may be set to a lower frequency then communications during daytime hours.

There may also be location-specific conditions that dictate the type and frequency of communications. For example, the current location of the patient, such as whether the patient is located at home or within the hospital, may change the types and frequency of messages that are transmitted to the patient's caregiver. As one non-limiting example, when the user is determined to be at home, or otherwise physically located outside of a hospital, the analyte sensor control device 102 may be configured to communicate directly with remote application server 155 or the caregiver's device (e.g., multi-purpose data receiving device 130 or user device 135), such as when the user is located at home. In this manner, analyte data may be stored directly by the remote application server 155, such as in an EMR associated with the patient, and/or provided directly to the caregiver device even when the patient is located at his home.

Although the illustrated embodiments of the continuous analyte monitoring system 100 include only one of each of the illustrated devices, this disclosure contemplates the continuous analyte monitoring system 100 incorporate multiples of each components interacting throughout the system. For example and without limitation, as embodied herein, data reading device 120 and/or multi-purpose data receiving device 130 can include multiples of each. As embodied herein, multiple data receiving devices 130 can communicate directly with sensor control device 102 as described herein. Additionally or alternatively, a data receiving device 130 can communicate with secondary data receiving devices 130 to provide analyte data, or visualization or analysis of the data, for secondary display to the user or other authorized parties.

### Exemplary Reader Device

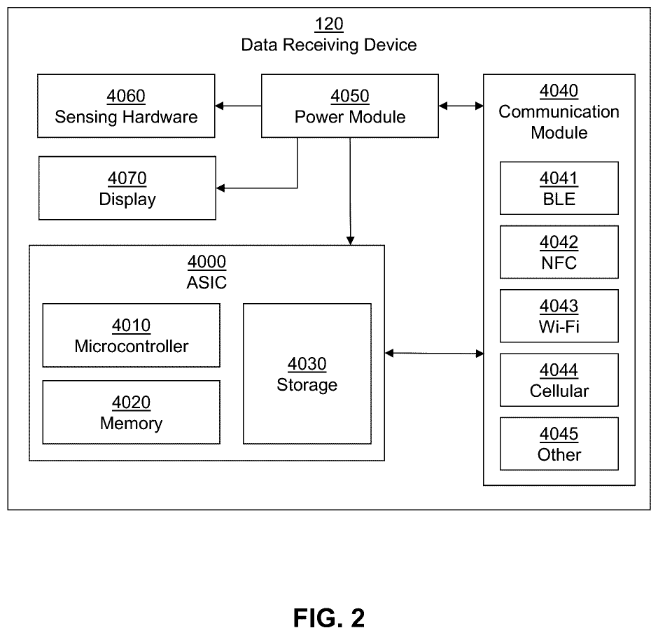

FIG. 2 is a block diagram depicting an example embodiment of a reader device configured as a smartphone. The following description will focus on data receiving device 120 but it is understood that the components and functionality may also apply to other reader devices, such as multi-purpose data receiving device 130 and user device 135. For purpose of illustration and not limitation, reference is made to the exemplary embodiment of data receiving device 120 for use with the disclosed subject matter as shown in FIG. 2. Data receiving device 120 and the related multi-purpose data receiving device 130 include components germane to the discussion of analyte sensor control device 102 and its operations and additional components can be included. In particular embodiments, data receiving device 120 and multi-purpose data receiving device 130 can be or include components provided by a third party and are not necessarily restricted to include devices made by the same manufacturer as analyte sensor control device 102.

As illustrated in FIG. 2, data receiving device 120 includes an ASIC 4000 having a microcontroller 4010, memory 4020, and storage 4030 and communicatively coupled with a communication module 4040. Power for the components of data receiving device 120 can be delivered by power module 4050, which as embodied herein can include a rechargeable battery. Data receiving device 120 can further include display 4070 for facilitating review of analyte data received from analyte sensor control device 102 or other device (e.g., user device 135 or remote application server 155). Data receiving device 120 can include separate user interface components (e.g., physical keys, light sensors, microphones, etc.).

Communication module 4040 can include Bluetooth low energy (BLE) module 4041 and NFC module 4042. Data receiving device 120 can be configured to wirelessly couple with analyte sensor control device 102 and transmit commands to and receive data from analyte sensor control device 102. As embodied herein, data receiving device 120 can be configured to operate, with respect to analyte sensor control device 102 as described herein, as an NFC scanner and a BLE end point via specific modules (e.g., BLE module 4042 or NFC module 4043) of communication module 4040. For example, data receiving device 120 can issue commands (e.g., activation commands for a data broadcast mode of the sensor; pairing commands to identify data receiving device 120) to analyte sensor control device 102 using a first module of the communication module 4040 and receive data from and transmit data to analyte sensor control device 102 using a second module of the communication module 4040.

As another example, communication module 4040 can include, for example, cellular radio module 4044. The cellular radio module 4044 can include one or more radio transceivers for communicating using broadband cellular networks, including, but not limited to third generation (“3G”), fourth generation (“4G”), and fifth generation (“5G”) networks. Additionally, communication module 4040 of data receiving device 120 can include Wi-Fi radio module 4043 for communication using a wireless local area network according to one or more of the IEEE 802.11 standards (e.g., 802.11a, 802.11b, 802.11 g, 802.11n (aka Wi-Fi 4), 802.11ac (aka Wi-Fi 5), 802.11ax (aka Wi-Fi 6)). Using cellular radio module 4044 or Wi-Fi radio module 4043, data receiving device 120 can communicate with remote application server 155 to receive analyte data or provide updates or input received from a user (e.g., through one or more user interfaces). Communication module 4040 can further include other protocol module 4045 that are compatible with additional wireless standards for Internet of Things (IoT) devices, such as Zigbee and Matter. In some embodiments, other protocol module 4045 can include additional or alternative chipsets for use with other short-range communication schemes, such as short-range radio (other than Bluetooth or BLE), personal area network according to IEEE 802.15 protocols, IEEE 802.11 protocols, infrared communications according to the Infrared Data Association standards (IrDA), etc.

In some embodiments, other protocol module 4045 may also be configured to be compatible with proprietary protocols such as proprietary short-range radio protocols that do not include Bluetooth or BLE. One non-limiting example is the Medical Implement Communication System (MICS) protocol which is configured for transmitting data between medical devices implanted in a patient. MICS protocol operates at a frequency from 402 to 405 MHz. Being compatible with other short-range radio protocols could be particular advantageous in a hospital setting where Bluetooth and/or BLE can potentially interfere with operations and communications with medical devices that are not intended recipients of Bluetooth and/or BLE messages. Other short-range radio protocols avoid this interference issue. Additionally, proprietary short-range radio protocols may also offer more security as Bluetooth and/or BLE can be susceptible to cyberattacks.

As embodied herein, on-board storage 4030 of data receiving device 120 can store analyte data received from analyte sensor control device 102. Further, data receiving device 120, multi-purpose data receiving device 130, or user device 135 can be configured to communicate with remote application server 155 via a wide area network. As embodied herein, analyte sensor control device 102 can provide data to data receiving device 120 or multi-purpose data receiving device 130. Data receiving device 120 can transmit the data to user device 135. User device 135 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor control device 102. In particular embodiments, data receiving device 120 can be configured to operate in coordination with analyte sensor control device 102 and based on analyte data received from the analyte sensor control device 102. As an example, where analyte sensor control device 102 is a glucose sensor, data receiving device 120 can be or include a medication delivery device, such as an insulin pump or insulin injection pen. In coordination, multi-purpose data receiving device 130 can adjust an insulin dosage for a user based on glucose values received from the analyte sensor.

### Exemplary Sensor Devices

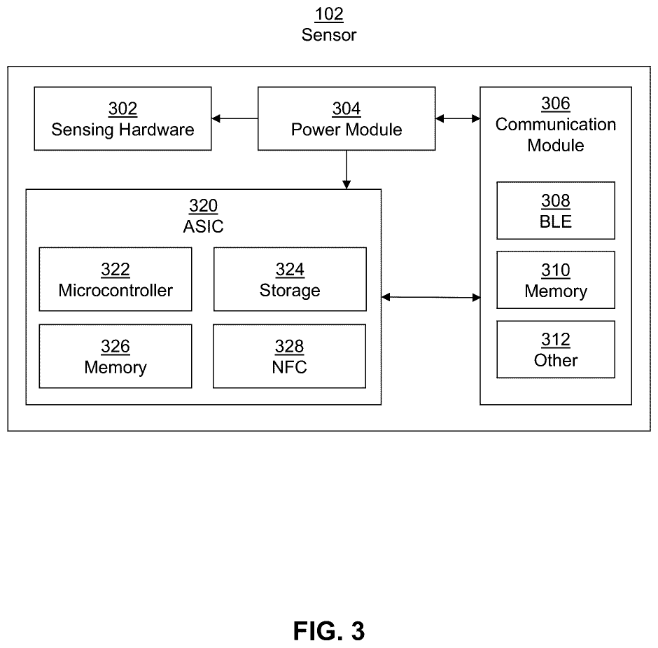

FIG. 3 is a block diagram depicting example embodiments of sensor control device 102. For purpose of illustration and not limitation, reference is made to the exemplary embodiment of analyte sensor control device 102 for use with the disclosed subject matter as shown in FIG. 3. FIG. 3 illustrates a block diagram of an example analyte sensor control device 102 according to exemplary embodiments compatible with the security architecture and communication schemes described herein.

As embodied herein, analyte sensor control device 102 can include ASIC 320 communicatively coupled with a communication module 306. ASIC 320 can include a microcontroller core 322, on-board memory 326, and storage memory 324. Storage memory 324 can store data used in an authentication and encryption security architecture. Storage memory 324 can store programming instructions for analyte sensor control device 102. As embodied herein, certain communication chipsets can be embedded in the ASIC 320 (e.g., an NFC transceiver 328). ASIC 320 can receive power from a power module 5050, such as an on-board battery or from an NFC pulse. Storage memory 324 of the ASIC 320 can be programmed to include information such as an identifier for analyte sensor control device 102 for identification and tracking purposes. Storage memory 324 can also be programmed with configuration or calibration parameters for use by analyte sensor control device 102 and its various components. Storage memory 324 can include rewritable or one-time programming (“OTP”) memory. The storage memory 324 can be updated using techniques described herein to extend the usefulness of analyte sensor control device 102.

To perform its functionalities, sensor control device 102 can further include suitable sensing hardware 302 appropriate to its function. As embodied herein, sensing hardware 302 can include an analyte sensor transcutaneously or subcutaneously positioned in contact with a bodily fluid of a subject. The analyte sensor can generate sensor data containing values corresponding to levels of one or more analytes within the bodily fluid.

The storage memory 324 of the sensor control device 102 can include the software blocks related to communication protocols of the communication module. For example, the storage memory 324 can include a BLE services software block with functions to provide interfaces to make the BLE module 308 available to the computing hardware of the sensor control device 102. These software functions can include a BLE logical interface and interface parser. BLE services offered by the communication module 306 can include the generic access profile service, the generic attribute service, generic access service, device information service, data transmission services, and security services. The data transmission service can be a primary service used for transmitting data such as sensor control data, sensor status data, analyte measurement data (historical and current), and event log data. The sensor status data can include error data, current time active, and software state. The analyte measurement data can include information such as current and historical raw measurement values, current and historical values after processing using an appropriate algorithm or model, projections and trends of measurement levels, comparisons of other values to patient-specific averages, calls to action as determined by the algorithms or models and other similar types of data.

As embodied herein, communication module 306 of sensor control device 102 can be implemented as or include one or more modules to support analyte sensor control device 102 communicating with other devices of the continuous analyte monitoring system 100. As an example only and not by way of limitation, example communication module 306 can include BLE module 308, memory 310, and other protocol module 312. As used throughout this disclosure, BLE refers to a short-range communication protocol optimized to make pairing of Bluetooth devices simple for end users. Communication module 306 can transmit and receive data and commands via interaction with similarly-capable communication modules of data receiving device 120 or user device 135. Communication module 306 may store the data and commands in memory 310.

Communication module 306 can include an additional module 312 for other protocols and/or additional or alternative chipsets for use with similar short-range communication schemes, such as a personal area network according to IEEE 802.15 protocols, IEEE 802.11 protocols, infrared communications according to the Infrared Data Association standards (IrDA), etc.

According to aspects of the disclosed subject matter, and as embodied herein, a sensor control device 102 can be configured to communicate with multiple devices concurrently by adapting the features of a communication protocol or medium supported by the hardware and radios of the sensor control device 102. As an example, the BLE module 308 of the communication module 306 can be provided with software or firmware to enable multiple concurrent connections between the sensor control device 102 as a central device and the other devices such as anyone of data receiving device 120, multi-purpose data receiving device 130, and/or user device 135.

Connections, and ensuing communication sessions, between two devices using a communication protocol such as BLE can be characterized by a similar physical channel operated between the two devices (e.g., a sensor control device 102 and data receiving device 120). The physical channel can include a single channel or a series of channels, including for example and without limitation using an agreed upon series of channels determined by a common clock and channel—or frequency-hopping sequence.

Communication sessions can use a similar amount of the available communication spectrum, and multiple such communication sessions can exist in proximity. In certain embodiment, each collection of devices in a communication session uses a different physical channel or series of channels, to manage interference of devices in the same proximity.

For purpose of illustration and not limitation, reference is made to an exemplary embodiment of a procedure for a sensor-receiver connection for use with the disclosed subject matter. First, the sensor control device 102 repeatedly advertises its connection information to its environment in a search for a data receiving device 120. The sensor control device 102 can repeat advertising on a regular basis until a connection established. The data receiving device 120 detects the advertising packet and scans and filters for the sensor control device 102 to connect to through the data provided in the advertising packet. Next, data receiving device 120 sends a scan request command and the sensor control device 102 responds with a scan response packet providing additional details. Then, the data receiving device 120 sends a connection request using the Bluetooth device address associated with the data receiving device 102. The data receiving device 120 can also continuously request to establish a connection to a sensor control device 102 with a specific Bluetooth device address. Then, the devices establish an initial connection allowing them to begin to exchange data. The devices begin a process to initialize data exchange services and perform a mutual authentication procedure.

During a first connection between the sensor control device 102 and data receiving device 120, the data receiving device 120 can initialize a service, characteristic, and attribute discovery procedure. The data receiving device 120 can evaluate these features of the sensor control device 102 and store them for use during subsequent connections. Next, the devices enable a notification for a customized security service used for mutual authentication of the sensor control device 102 and data receiving device 120. The mutual authentication procedure can be automated and require no user interaction. Following the successful completion of the mutual authentication procedure, the sensor control device 102 sends a connection parameter update to request the data receiving device 120 to use connection parameter settings preferred by the sensor control device 102 and configured to maximum longevity.

In some embodiments, the data receiving device 120 then performs sensor control procedures to backfill historical data, current data, event log, and factory data. As an example, for each type of data, the data receiving device 120 sends a request to initiate a backfill process. The request can specify a range of records defined based on, for example, the measurement value, timestamp, or similar, as appropriate. The sensor control device 102 responds with requested data until all previously unsent data in the memory of the sensor control device 102 is delivered to the data receiving device 120. The sensor control device 102 can respond to a backfill request from the data receiving device 120 that all data has already been sent. Once backfill is completed, the data receiving device 120 can notify sensor control device 102 that it is ready to receive regular measurement readings. The sensor control device 102 can send readings across multiple notifications result on a repeating basis. As embodied herein, the multiple notifications can be redundant notifications to ensure that data is transmitted correctly. Alternatively, multiple notifications can make up a single payload.

As embodied herein, certain calibration features for the sensing hardware 302 of the analyte sensor control device 102 can be adjusted based on external or interval environment features as well as to compensate for the decay of the sensing hardware 302 during expended period of disuse (e.g., a “shelf time” prior to use). The calibration features of the sensing hardware 302 can be autonomously adjusted by the sensor control device 102 (e.g., by operation of the ASIC 320 to modify features in the memory 326 or storage memory 324) or can be adjusted by other devices of the CAM system 100.

As an example, sensor sensitivity of the sensing hardware 302 can be adjusted based on external temperature data or the time since manufacture. When external temperatures are monitored during the storage of the sensors, the disclosed subject matter can adaptively change the compensation to sensor sensitivity over time when the device experiences changing storage conditions. For purpose of illustration not limitations, adaptive sensitivity adjustment can be performed in an “active” storage mode where the analyte sensor control device 102 wakes up periodically to measure temperature. These features can save the battery of the analyte device and extend the lifespan of the analyte sensors. At each temperature measurement, the analyte sensor control device 102 can calculate a sensitivity adjustment for that time period based on the measured temperature. Then, the temperature-weighted adjustments can be accumulated over the active storage mode period to calculate a total sensor sensitivity adjustment value at the end of the active storage mode (e.g., at insertion). Similarly, at insertion, the analyte sensor control device 102 can determine the time difference between manufacture of the sensor control device 102 (which can be written to the storage 324 of the ASIC 320) or the sensing hardware 302 and modify sensor sensitivity or other calibration features according to one or more known decay rates or formulas.

Additionally, for purpose of illustration and not limitation, as embodied herein, sensor sensitivity adjustments can account for other sensor conditions, such as sensor drift. Sensor sensitivity adjustments can be hardcoded into the analyte sensor control device 102 during manufacture, for example in the case of sensor drift, based on an estimate of how much an average sensor would drift. Analyte sensor control device 102 can use a calibration function that has time-varying functions for sensor offset and gain, which can account for drift over a wear period of the sensor. Thus, analyte sensor control device 102 can utilize a function used to transform an interstitial current to interstitial glucose utilizing device-dependent functions describing analyte sensor control device 102 drift over time, and which can represent sensor sensitivity, and can be device specific, combined with a baseline of the glucose profile. Such functions to account for sensor sensitivity and drift can improve analyte sensor control device 102 accuracy over a wear period and without involving user calibration.

In some embodiments, the sensor control device 102 detects raw measurement values from sensing hardware 302. On-sensor processing can be performed, such as by one or more models trained to interpret the raw measurement values. Models can be machine learning models trained off-device to detect, predict, or interpret the raw measurement values to detect, predict, or interpret the levels of one or more analytes. Machine learning model may be used to predict future analyte levels, and/or to predict future analyte level events, such as high or low analyte levels as well as forecasting analyte trends within the patient analyte data. Additional trained models can operate on the output of the machine learning models trained to interact with raw measurement values. As an example, models can be used to detect, predict, or recommend events based on the raw measurements and type of analyte(s) detected by the sensing hardware 302. Events can include, initiation or completion of physical activity, meals, application of medical treatment or medication, emergent health events, and other events of a similar nature.

Models can be provided to any combination of the sensor control device 102, data receiving device 120, or multi-purpose data receiving device 130 during manufacture or during firmware or software updates. Models can be periodically refined, such as by the manufacturer of the sensor control device 102 or the operator of the CAM system 100, based on data received from the sensor control device 102 and data receiving devices of an individual user or multiple users collectively. In certain embodiments, the sensor control device 102 includes sufficient computational components to assist with further training or refinement of the machine learning models, such as based on unique features of the user to which the sensor control device 102 is attached. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learning models. Model-based processing can be performed by other devices, including the data receiving device 120 or multi-purpose data receiving device 130, upon receiving data from the sensor control device 102 (or other downstream devices).

When applied to continuous lactate monitoring, machine learning models may rely on the real-time data being provided by, for example, sensor control device 102, to continuously train the model or update the training set for the model. Machine learning models of the present disclosure may also be trained using patient vital information, patient medical history, and in some embodiments, cohort or population information, which would increase the size of the training set to include lactate and medical information from patients that are similar to the current patient.

Data transmitted between the sensor control device 102 and any one of a data receiving device 120, multi-purpose data receiving device 130, and/or user device 135 can include raw or processed measurement values. Data transmitted between the sensor control device 102 and any one of a data receiving device 120, multi-purpose data receiving device 130, and/or user device 135 can further include alarms or notification for display to a user. The data receiving device 120, multi-purpose data receiving device 130, and/or user device 135 can display or otherwise convey notifications to the user based on the raw or processed measurement values or can display alarms when received from the sensor 110. Alarms may be triggered for display to the user based on one or more predictions associated with the patient. Alarms may include one or more of the following alarms and various combinations thereof. An alarm may be based on the predicted patient outcome (e.g., when compared to different levels of severity thresholds), direct analyte values (e.g., one-time reading exceeding a threshold or failing to satisfy a threshold), analyte value trends (e.g., average reading over a set period of time exceeding a threshold or failing to satisfy a threshold; slope); analyte value predictions (e.g., algorithmic calculation based on analyte values exceeds a threshold or fails to satisfy a threshold), sensor alerts (e.g., suspected malfunction detected), communication alerts (e.g., no communication between sensor control device 102 and data receiving device 120 for a threshold period of time; unknown device attempting or failing to initiate a communication session with the sensor control device 102), reminders (e.g., reminder to charge data receiving device 120; reminder to take a medication or perform other activity), and other alerts of a similar nature. For purpose of illustration and not limitation, as embodied herein, the alarm parameters described herein can be configurable by a user or can be fixed during manufacture, or combinations of user-settable and non-user-settable parameters.

### Exemplary Over-the-Air Updates

FIG. 1 further illustrates an example operating environment for providing over-the-air (“OTA”) updates for use with the techniques described herein. An operator of continuous analyte monitoring system 100 can bundle updates for data receiving device 120 or analyte sensor control device 102 into updates for an application executing on multi-purpose data receiving device 130. Using available communication channels between data receiving device 120, multi-purpose data receiving device 130, and analyte sensor control device 102, multi-purpose data receiving device 130 can receive regular updates for data receiving device 120 or analyte sensor control device 102 and initiate installation of the updates on data receiving device 120 or analyte sensor control device 102. Multi-purpose data receiving device 130 acts as an installation or update platform for data receiving device 120 or analyte sensor control device 102 because the application that enables the multi-purpose data receiving device 130 to communicate with analyte sensor control device 102, data receiving device 120 and/or remote application server 155 can update software or firmware on data receiving device 120 or analyte sensor control device 102 without wide-area networking capabilities.

As embodied herein, remote application server 155 operated by the manufacturer of analyte sensor control device 102 and/or the operator of continuous analyte monitoring system 100 can provide software and firmware updates to the devices of continuous analyte monitoring system 100. In particular embodiments, remote application server 155 can provides the updated software and firmware to user device 135 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor control device 102, data receiving device 120 or analyte sensor control device 102 and multi-purpose data receiving device 130 establish a connection. Multi-purpose data receiving device 130 determines that a firmware or software update is available for data receiving device 120 or analyte sensor control device 102. Multi-purpose data receiving device 130 can prepare the software or firmware update for delivery to data receiving device 120 or analyte sensor control device 102. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor control device 102. Multi-purpose data receiving device 130 can also send a command to data receiving device 120 or analyte sensor control device 102 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor control device 102 receives the data for the update and the command to initiate the update from multi-purpose data receiving device 130. Data receiving device 120 can then install the firmware or software update. To install the update, data receiving device 120 or analyte sensor control device 102 can place or restart itself in a so-called “safe” mode with limited operational capabilities. Once the update is completed, data receiving device 120 or analyte sensor control device 102 re-enters or resets into a standard operational mode. Data receiving device 120 or analyte sensor control device 102 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 324 of analyte sensor control device 102 includes OTP memory. The term OTP memory can refer to memory that includes access restrictions and security to facilitate writing to particular addresses or segments in the memory a predetermined number of times. Storage memory 324 can be prearranged into multiple pre-allocated memory blocks or containers. The containers are pre-allocated into a fixed size. If storage memory 324 is OTP memory, the containers can be considered to be in a non-programmable state. Additional containers which have not yet been written to can be placed into a programmable or writable state. Containerizing storage memory 324 in this fashion can improve the transportability of code and data to be written to storage memory 324. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

### Summary of Various Continuous Analyte Monitoring System Deployments

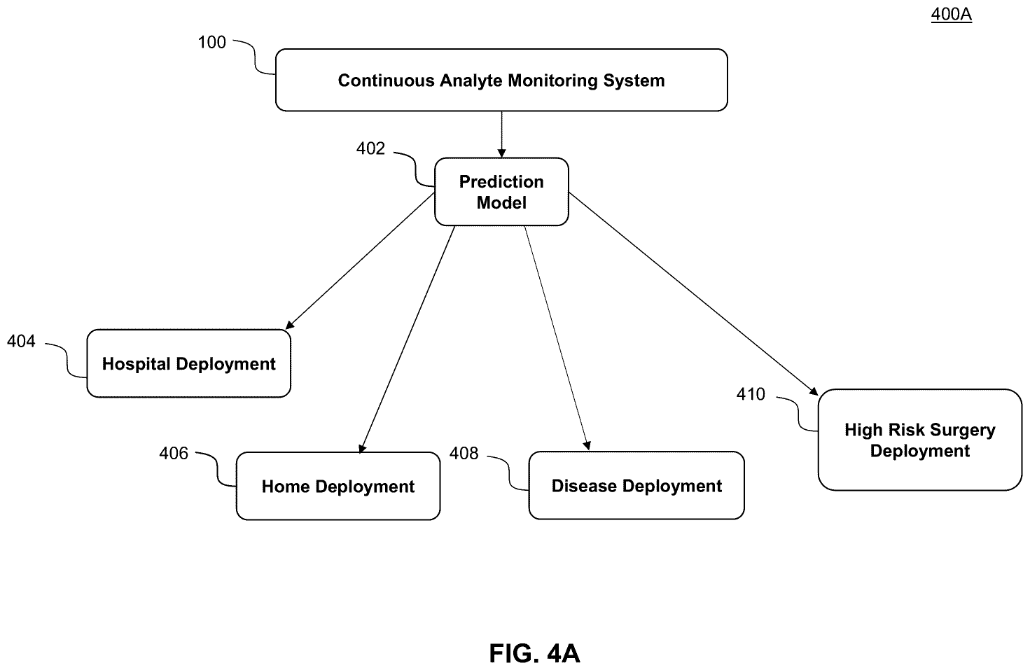

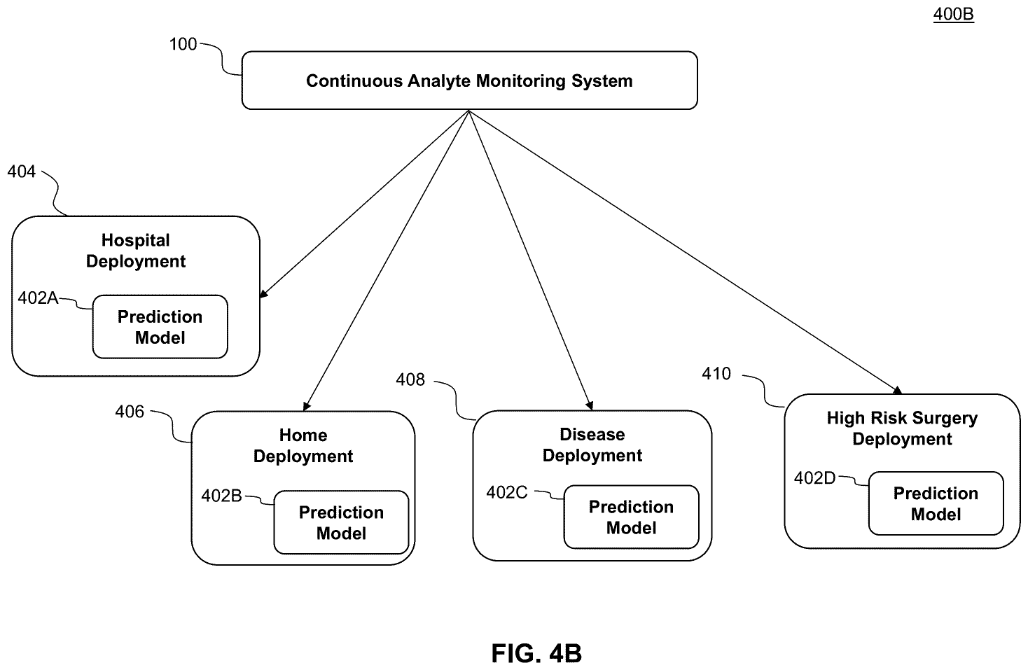

FIG. 4A is a block diagram of an early warning system 400A that includes continuous analyte monitoring (CAM) system 100 that may be deployed in various operating environments including one or more of a hospital deployment 404, home deployment 406, disease deployment 408, and high risk surgery deployment 410, according to some embodiments.

CAM system 100 may continuously monitor analyte levels of patients and provide the analyte data to a prediction model 402. Prediction model 402 is configured to process the analyte data and provide predictions of patient outcomes such as analyte trends or patient events for use in various clinical settings, such as one or more of hospital deployment 404, home deployment 406, disease deployment 408, or high risk surgery deployment 410. Prediction model 402 is not limited to only generating predictions of patient outcomes but may also generate recommended treatments, wherein the treatments may be based on predictions. For example, a predicted future analyte level is used to determine a recommendation for a medication dosage. In some embodiments, the predictions and/or the recommended treatments may be transmitted and displayed on one or more of data receiving device 120, multi-purpose data receiving device 130, user device 135 and/or remote application server 155. In some embodiments, prediction model 402 may provide the predictions to CAM system 100 for dynamic and automatic execution of treatment, such as by transmitting commands to treatment devices connected to CAM system 100.

Although only one prediction model 402 is depicted in FIG. 4A, CAM system 100 may utilize more than one, in a distributed manner. For example, FIG. 4B depicts an early warning system 400B that includes a prediction model generated and trained for each type of deployment, with prediction model 402A dedicated for generating predictions for hospital deployment 404, prediction model 402B for generating predictions for home deployment 406, prediction model 402C for generating predictions for disease detection deployment 408, and prediction model 402D for high risk surgeries deployment 410. Prediction models are configured to provide predictions for patient outcomes based on analyte data for a patient.

In some embodiments, prediction model 402A implemented in a hospital deployment 404 may include one or more prediction models implemented at respective hospital locations, such that prediction model 402A may be trained on data specific to each hospital and therefore provide predictions that are more specific to each hospital to which the prediction model 402A is deployed. Further still, in some embodiments, prediction model 402A may comprise multiple prediction models within hospital deployment 404, with each prediction model being implemented on a per-patient (or per-department) basis within hospital deployment 404. Each prediction model may be trained on data specific to each patient (or department) to provide predictions that are more specific to each patient (or department) within the hospital deployment 404.

Prediction model 402B may be implemented on a per-patient basis (e.g., installed on a device associated with the patient such as data receiving device 120, multi-purpose data receiving device 130, and/or user device 135) and trained with data specific to each patient and to the home deployment 406. Prediction model 402B may therefore differ in its training than prediction model 402A, and is trained to generate predictions that are more tailored for each patient and the home deployment 406.

Prediction model 402C may be implemented on a per-disease basis (e.g., sepsis, heart failure) and trained with data specific to each disease within disease deployment 408. Prediction model 402C may therefore be trained to generate predictions that are more tailored for each disease. Prediction model 402D may be implemented for specific high risk surgeries and trained to generate predictions that are more tailored for each surgery.

In another example embodiment, artificial intelligence (AI), such as machine-learning (ML) systems train the prediction model (e.g., prediction model 402 in FIG. 4A, prediction models 402A, 402B, 402C, and/or 402D in FIG. 4B) to generate more accurate predictions of patient outcomes based on analyte data and other patient information provided by other medical devices associated with patients. The prediction model may be continuously updated by future actual patient outcomes to determine the accuracy of prior outcome predictions and continued training of the prediction model so as to improve the accuracy of future predictions. ML involves computers discovering how they can perform tasks without being explicitly programmed to do so. ML includes, but is not limited to, artificial intelligence, deep learning, fuzzy learning, supervised learning, unsupervised learning, etc. Machine learning algorithms may build an initial prediction model based on sample data, known as “training data”, in order to make predictions or decisions without being explicitly programmed to do so. This sample data includes analyte data from any number of patients and may be provided by any number of CAM systems. For supervised learning, the computer is presented with example inputs and their desired outputs and the goal is to learn a general rule that maps inputs to outputs. In another example, for unsupervised learning, no labels are given to the learning algorithm, leaving it on its own to find structure in its input. Unsupervised learning can be the ultimate goal such as discovering trends or patterns in data, or a means towards another goal, such as improved accuracy of future predictions.

A machine-learning engine may use various classifiers to map concepts or data provided by different CAM systems and/or different deployment to capture relationships between concepts (e.g., analyte levels and patient outcomes in different deployments) and an accuracy of prior predicted patient outcomes. The classifier (discriminator) is trained to distinguish (recognize) in variations of data from different patients, CAM systems, and/or different deployments.

In some aspects, machine learning models are trained on a remote machine learning platform using a history of analyte data and patient information from one or more other patients, CAM systems, and/or different deployments. In addition, collecting patient data into large training sets may allow for the training data to be normalized (e.g., not skewed by a single or few occurrences of a data artifact). In one embodiment, prediction models are continuously updated as new patient information is received.

Hospital deployment 404 refers to utilizing analyte data (e.g., continuous lactate, creatinine, potassium, and/or glucose information) from a patient to predict the patient's medical outcome when the patient is located within a hospital setting. A CAM system 100 implemented within a hospital deployment 404 may be configured with devices that are typically utilized within the hospital setting. For example, any one of data receiving device 120, multi-purpose data receiving device 130, and user device 135 may be implemented as any one of a patient bedside monitoring device, one or more computers at a nursing station, and one or more handheld devices carried by caregivers in the hospital setting. In some embodiments, any number and combination of data receiving device 120, multi-purpose data receiving device 130, and user device 135 may be implemented as a patient bedside monitoring ecosystem with each device implemented in the ecosystem capable of transmitting and receiving analyte data and other patient data from other devices. Devices in the patient bedside monitoring ecosystem may be configured to synchronize data with each other.

Home deployment 406 refers to utilizing analyte data (e.g., continuous lactate and/or glucose information) from a patient to predict the patient's medical outcome when the patient is located within a home setting. A CAM system 100 implemented within a home deployment 406 may be configured with devices that are typically utilized within the home setting. For example, any one of data receiving device 120, multi-purpose data receiving device 130, and user device 135 may be implemented as any one of a patient bedside monitoring device and one or more handheld devices carried by caregivers of the patient for receiving updates about their patients.

Disease deployment 408 refers to utilizing analyte data (e.g., continuous lactate and/or glucose information) from a patient to predict the patient's medical outcome for different disease detection use cases, including sepsis and heart failure.

High risk surgery deployment 410 refers to utilizing analyte data (e.g., continuous lactate and/or glucose information) from a patient to predict the patient's medical outcome when the patient has undergone high-risk surgeries.

### Hospital Deployment

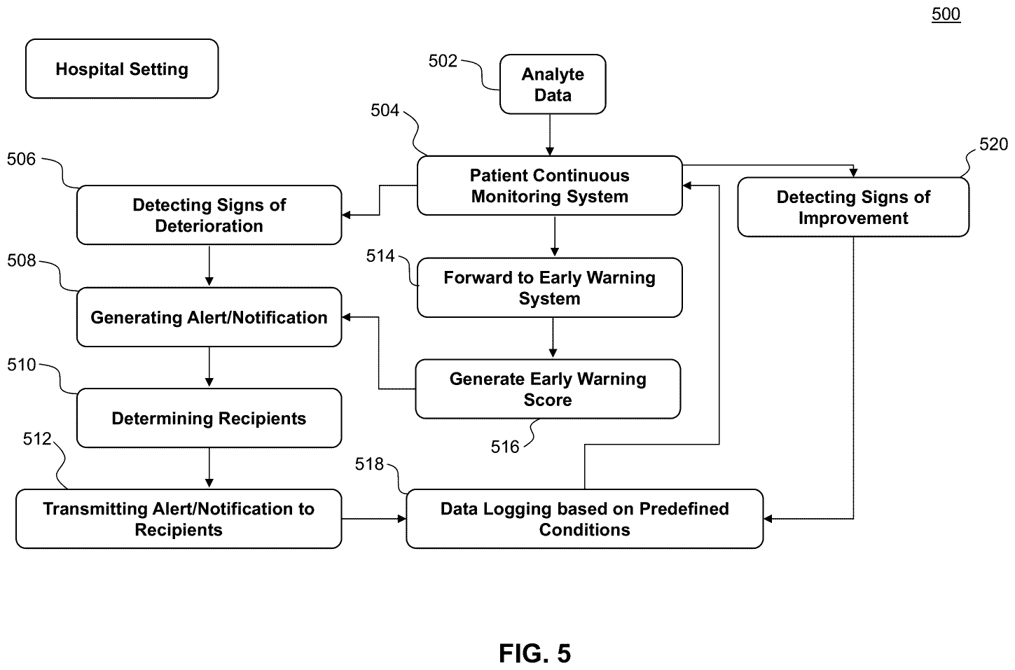

FIG. 5 is a flowchart illustrating a method 500 for utilizing a continuous analyte monitoring system within a hospital environment, according to some embodiments. Method 500 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 5 may be performed by a components of CAM system 100, either separately or in a distributed fashion. In such an embodiment, any of these components may execute code in memory to perform certain steps of method 500 of FIG. 5. While method 500 of FIG. 5 will be discussed below as being performed by certain components of CAM system 100, other components may store the code and therefore may execute method 500 by directly executing the code. Accordingly, the following discussion of method 500 will refer to components of FIGS. 1-3 as an exemplary non-limiting embodiment. Moreover, it is to be appreciated that not all steps may be needed to perform the disclosure provided herein. Further, some of the functions may be performed simultaneously, in a different order, or by the same components than shown in FIG. 5, as will be understood by a person of ordinary skill in the art.

Hospital admissions generally fall into two broad categories: a) acute care-medical and surgical emergencies and b) planned elective admissions, typically for planned surgery. CAM system 100 may be utilized during acute illnesses such as myocardial infarction, diabetic ketoacidosis, acute appendicitis to detect levels of lactate as part of generating and improving a prediction model for predicting patient medical outcome and for dynamically adjusting patient treatment based on monitoring patient response.

CAM system 100 is configured to continuously monitor lactate levels of any patient in a hospital setting. In some embodiments, analyte sensor control device 102 of CAM system 100 may be implemented as part of continuous lactate monitor system for measuring lactate levels in dermal interstitial fluid of patients which allows closer monitoring of the lactate levels of patients in the acute hospital setting. CAM system 100 also provides a novel means of measuring lactate levels in a clinical setting while providing an increased level of surveillance of a patient's lactate levels with the potential of reducing the requirement for multiple blood tests, allowing real-time monitoring of the patient's response to treatment, and allow the early identification of deterioration.

Continuous lactate monitoring could be used to monitor patients, screening for signs of deterioration. This would reduce the blind spot between nursing observations, potentially allowing for earlier identification of patient decline. This could lead to a reduction in patient mortality, reduction in intensive care admission, and shorten inpatient length of stay.

Analyte sensor control device 102 may be implemented as a biowearable analyte (e.g., lactate, glucose, dual analyte, or multiple analyte) sensor. In some embodiments, a dual-analyte sensor may comprise a sensor capable of detecting lactate and another analyte sensor. Examples of dual-analyte sensors include glucose-lactate and glucose-ketone. One example of a multiple analyte sensor includes glucose-lactate-ketone sensor. Other examples of analytes that may be implemented as part of a dual or multiple analyte sensor include glucose, ketones, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, or any combination thereof. Benefits of using a biowearable sensor include but are not limited to being less expensive, non-invasive/non-permanent, no procedure/no in-person visit, no expert team and equipment, easy start-up, easy data capture, reducing the need for sensor calibration, and easy data sharing with patient and provider. In some embodiments, calibration of the sensor may be implemented as an option. In some embodiments, the option of calibration may be used to supplement factory calibration, without requiring end-user calibration. Additionally, a biowearable sensor may be updated with new hardware and/or software to provide longer-term improvements including providing an accelerometer/altimeter for activities of daily living, providing heart rate and heart rate variability data capture, extending wear-time from 15 days to one month, and adding additional biomarkers including proteins.

In 502, analyte data associated with a patient is received (e.g., from analyte sensor control device 102). The analyte data may include any analyte including, but not limited to, lactate, glucose, or creatinine. Any combination of analytes may be utilized and may depend on the use-case deployment for which the predicted patient outcome is being provided. As one non-limiting example, creatinine may be input into the prediction model for predicting a length of stay for a patient after cardiac and/or renal surgery.

In 504, the analyte data is provided between components in a patient continuous monitoring system (e.g., between analyte sensor control device 102 and any combination of data receiving device 120, multi-purpose data receiving device 130, and/or user device 135). CAM system 100 is an exemplary implementation of a patient continuous monitoring system. A component (any combination of data receiving device 120, multi-purpose data receiving device 130, and/or user device 135) in CAM system 100 may also receive other information associated with patient including the patient's medical information and trend information (e.g., blood pressure, heart rate, respiratory rate, treatment history including current and prior medicine). CAM system 100 provides the analyte and medical information to a prediction model for generating a predicted patient outcome based on the analyte and medical information. In some embodiments, the prediction model may be implemented in any combination of data receiving device 120, multi-purpose data receiving device 130, and/or user device 135, or on a per-deployment basis such as prediction models 402A-D discussed with respect to FIG. 4B. Examples of a predicted patient outcome include, but are not limited to, upward or downward trends in patient condition (i.e., patient deterioration), patient response to potential treatment, length of stay following a procedure, and indicators of a potential disease or condition. For example, the prediction model may be trained to predict the length of a patient's stay following a procedure based on the patient's continuous lactate information.

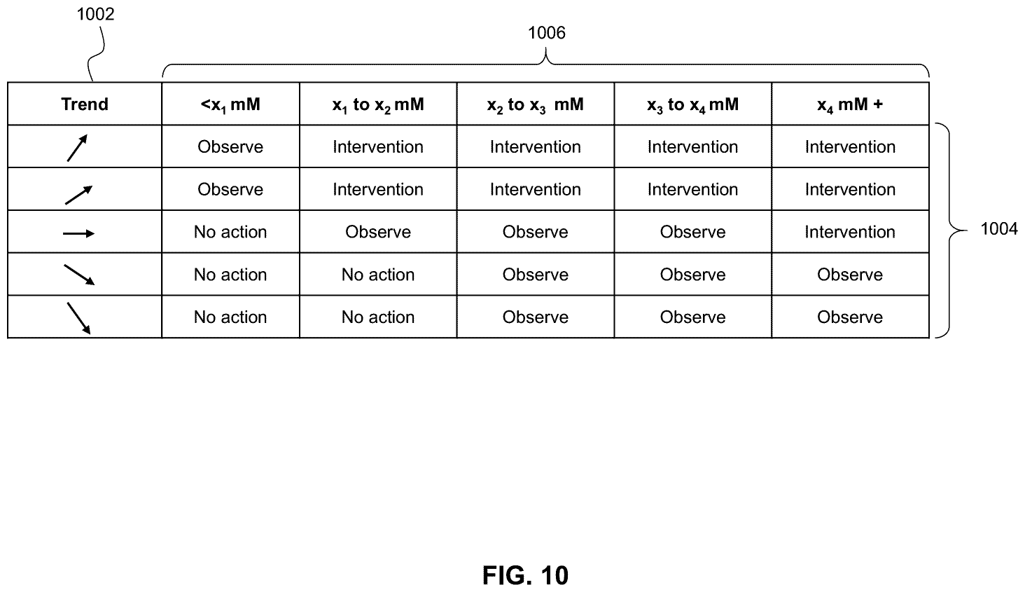

In 506, output of the prediction model may be used by CAM system 100 to detect signs of deterioration in the patient. For example, a prediction model may be implemented in any combination of data receiving device 120, multi-purpose data receiving device 130, and/or user device 135. Each device may be configured to generate a predicted patient outcome based on received analyte data from analyte sensor control device 102 associated one or more patients. In some aspects, the device may also receive medical information associated the one or more patients and may be further configured to generate the predicted patient outcome based on both the analyte data and the medical information. In some aspects, detection may be based on comparing the predicted patient outcome to predefined threshold values and signs of deterioration may be determined based on the predicted patient outcome exceeding one or more predefined threshold values. In some aspects, the predicted patient outcome may be implemented as any combination of a number value (e.g., between one and a hundred), a text message (e.g., “suspected sepsis” or “patient vitals deteriorating”), a visual display such as a trend graph or a table (e.g., showing patient vitals including the continuously monitored analyte data). FIG. 10 depicts an exemplary trend table, which is discussed in further detail below.

In 508, a component in CAM system 100 (e.g., any combination of data receiving device 120, multi-purpose data receiving device 130, and/or user device 135) may generate an alert and/or notification based on the detection of any signs of deterioration in the patient. Alerts and notifications include the predicted patient outcome. An alert differs from a notification with regard to any required action that may be required based on the predicted patient outcome. For example, an alert may require an immediate action or treatment to be administered, either manually by a caregiver or automatically, by a medical device that is already connected to the patient. The alert may include one or more treatment recommendations for addressing the predicted patient outcome. In contrast, a notification may simply provide the predicted patient outcome and does not require any immediate action or treatment. Accordingly, alerts and notifications may be used based on a severity of the predicted patient outcome. CAM system 100 may generate the treatment recommendations based on the predicted patient outcome generated in 506. An alert and/or notification may be generated to notify one or more recipients of the patient's predicted outcome.

In 510, a component in CAM system 100 (e.g., any combination of data receiving device 120, multi-purpose data receiving device 130, and/or user device 135) may determine the recipients for receiving the generated alert and/or notification. Examples of recipients include health care providers such as nurses and doctors, as well as designated caregivers. The recipients may be predetermined based on different predicted patient outcomes. For example, if the predicted patient outcome identifies a particular disease (e.g., heart failure, sepsis), CAM system 100 may identify a predetermined recipient based on the identified disease. As another example, if the predicted patient outcome identifies a downward trend in the patient's condition, CAM system 100 may identify the predetermined recipient based on the severity of the downward trend (e.g., a first threshold level may trigger an alert and/or notification to a nurse and a second threshold level may trigger an alert and/or notification to the doctor).

In some embodiments, CAM system 100 implements an exemplary trend table 1000 as depicted in FIG. 10. For example, column 1002 can reflect varying trend rates (e.g., rapid rise, rise, no change, decline, rapid decline), rows 1004 can reflect recommended action items to be performed by CAM system 100, and columns 1006 can reflect analyte values that, in combination with trend rates from column 1002, can be used as inputs for detecting the appropriate action (or that no action is necessary).

In some embodiments, trend table 1000 may be generated on a patient specific basis such that each trend table 1000 may be associated with a unique patient identifier. In some embodiments, trend table 1000 may be dynamically updated as needed by a machine learning model (such as prediction model 402) based on patient specific data (such as past medical history, current medical history, inputs from an electronic health record) and user input (such as inputs received from a doctor's user device).

In 512, the alert and/or notification is transmitted to a device associated with the predetermined recipient (e.g., as indicated by recommended actions in rows 1004 from trend table 1000). Examples of devices associated with include their personal devices (with dedicated software installed) for receiving the alert and/or notification.

In 514, the generated predicted patient outcome may be forwarded to an early warning system. Some hospitals use an early warning score to screen patients for evidence of deterioration or signs of improvement. Early warning scores are typically based on the patient's vital signs such as heart rate, blood pressure and respiratory rate. CAM system 100 improves the accuracy of predicting deterioration in patients and transmission of the alerts by combining the patient's analyte data with an early warning score system.

In 516, CAM system 100 generates a dynamic early warning score based on any combination of the patient's analyte data (provided by a continuous analyte monitoring sensor) and the patient's medical information, including vital signs and trend information. For example, column 1004 of trend table 1000 in FIG. 10 indicates potential notifications or actions based on trend data in column 1002 and analyte data in columns 1006. Examples of notifications include “no action” (e.g., the patient's information indicates normal conditions), “observe” (e.g., the patient's information indicates potential issues) and “intervention” (e.g., sending notifications or alerts to determined recipients, including predetermined actions such as fluid resuscitation, antibiotics, or oxygen, just to name a few examples).

Trend information (e.g., from column 1002 of trend table 1000 in FIG. 10) refers to trends in the patient's analyte and medical history over a certain period of time (e.g., 8 hours, 12 hours). A dynamic early warning score differs from a conventional early warning score because it may be continuously updated based on continuously monitored analyte data from the patient as well as the patient's medical information.

Based on the dynamic early warning score (e.g., rows 1004 of trend table 1000 in FIG. 10), CAM system 100 may generate an alert and/or notification in 508. For example, if the dynamic early warning score is greater than predefined thresholds, CAM system 100 may be configured to generate one or more alerts and/or notifications to different caregivers in the hospital (e.g., as indicated by recommended actions in rows 1004 from trend table 1000).

In 518, remote application server 155 may receive and store the patient information in a remote database, such as in the patient's EMR. The patient information may be transmitted (e.g., by any combination of data receiving device 120, multi-purpose data receiving device 130, and/or user device 135) to remote application server based on any combination of predefined conditions, such as a schedule (e.g., daily, weekly), thresholds (e.g., if a dynamic early warning score exceeds an alert threshold) and the predicted patient outcome (e.g., if the predicted outcome exceeds a severity level). In 520, output of the prediction model may be used by CAM system 100 to detect signs of improvement in the patient. In some aspects, detection may be based on comparing the predicted patient outcome to predefined threshold values and signs of improvement may be determined based on the predicted patient outcome exceeding one or more predefined threshold values.

### Home Deployment

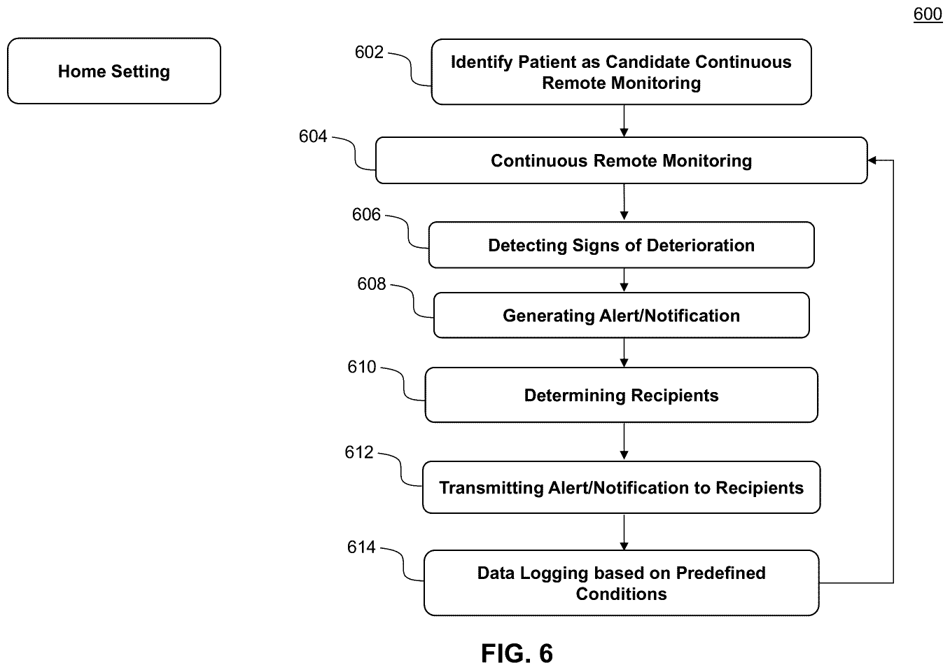

FIG. 6 is a flowchart illustrating a method 600 for utilizing a continuous analyte monitoring system within a home environment, according to some embodiments. Method 600 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 6 may be performed by a components of CAM system 100, either separately or in a distributed fashion. In such an embodiment, any of these components may execute code in memory to perform certain steps of method 600 of FIG. 6. While method 600 of FIG. 6 will be discussed below as being performed by certain components of CAM system 100, other components may store the code and therefore may execute method 600 by directly executing the code. Accordingly, the following discussion of method 600 will refer to components of FIGS. 1-3 as an exemplary non-limiting embodiment. Moreover, it is to be appreciated that not all steps may be needed to perform the disclosure provided herein. Further, some of the functions may be performed simultaneously, in a different order, or by the same components than shown in FIG. 6, as will be understood by a person of ordinary skill in the art.

Continuous lactate monitoring by CAM system 100 may be used to monitor patients in the home setting for signs of deterioration, thereby allowing earlier identification and proactive treatment to prevent hospitalization and deterioration. Measurement of lactate levels may be provided to a prediction model for providing an increased level of surveillance of a patient's lactate levels while the patient is at home. CAM system 100 may be configured to coordinate analyte data to a healthcare practitioner for early identification of deterioration and earlier intervention even when the patient is outside of the hospital setting.

Following discharge from hospital, or in patients identified at risk of deterioration in the home or long-term care setting, continuous analyte monitoring by CAM system 100 could be used to screen for signs of deterioration. Under the remote supervision of a healthcare professional, this could allow the earlier detection of patient decline leading to earlier intervention, carrying the potential to reduce patient mortality.

In 602, CAM system 100 receives patient identification of any patients that are identified candidates for continuous remote monitoring outside of the hospital. For example, during discharge from a hospital or during a visit to the doctor's visit, an HCP may determine that the patient is at risk for certain conditions that would merit continuous remote monitoring including the continuous remote monitoring of a patient's analyte levels. Once identified, a patient identifier associated with the patient may be CAM system 100 to indicate that the analyte sensor control device 102 associated with the patient is authorized to communicate remotely with components of the CAM system 100.

In 604, a component of CAM system 100 processes analyte data from the patient. For example, analyte sensor control device 102 associated with the patient may provide analyte data to any combination of data receiving device 120, multi-purpose data receiving device 130, and/or user device 135. In the home setting, analyte sensor control device 102 may be physically located in a different location and/or building than one or more components of CAM system 100. In some aspects, analyte sensor control device 102 is configured to communicate with multi-purpose data receiving device 130 which is then further configured to relay the analyte data from analyte sensor to one or more other components in CAM system 100 over a network, such as a Wi-Fi network or a cellular network. Examples of a multi-purpose data receiving device 130 in this embodiment include personal devices (e.g., mobile phone, tablet) associated with the patient with dedicated software for communicating with the analyte sensor control device 102. Accordingly, analyte sensor control device 102 may communicate analyte data to devices associated with designated recipients who may be located remotely from the patient. CAM system 100 may also process other information associated with patient including the patient's medical information and trend information (e.g., blood pressure, heart rate, respiratory rate, treatment history including current and prior medicine).

CAM system 100 provides the analyte and medical information to a prediction model for generating a predicted patient outcome based on the analyte and medical information. In some embodiments, prediction model may be implemented in any combination of data receiving device 120, multi-purpose data receiving device 130, and/or user device 135, which may be configured to generate a predicated patient outcome based on the analyte data, and in some additional embodiments, other medical information (e.g., patient's vitals).

In 606, output of the prediction model may be used by CAM system 100 to detect signs of deterioration in the patient. In some aspects, detection may be based on comparing the predicted patient outcome to predefined threshold values and signs of deterioration may be determined based on the predicted patient outcome exceeding one or more predefined threshold values.

In 608, a component of CAM system 100 (e.g., any combination of data receiving device 120, multi-purpose data receiving device 130, and/or user device 135) may generate an alert and/or notification based on the detection of any signs of deterioration in the patient. Alerts and notifications include the predicted patient outcome. The alert may include one or more treatment recommendations for addressing the predicted patient outcome. A component of CAM system 100 may generate the treatment recommendations based on the predicted patient outcome generated in 506 and include the generated recommendations in the alert. Accordingly, an alert and/or notification may be generated to notify one or more recipients of the patient's predicted outcome.

In 610, a component in CAM system 100 (e.g., any combination of data receiving device 120, multi-purpose data receiving device 130, and/or user device 135) may determine the recipients for receiving the generated alert and/or notification. The recipients may be predetermined based on different predicted patient outcomes, proximity to patient, time of day, and level of severity of the predicted outcome. When implemented in a home setting, the intended recipient may be identified based on their capability to more quickly respond to any alerts that require immediate action.

In 612, the alert and/or notification is transmitted to a device associated with the predetermined recipient. Examples of devices associated with include their personal devices (with dedicated software installed) for receiving the alert and/or notification.

In 614, remote application server 155 may receive and store the patient information including the predicted patient outcomes, the signs of deterioration, any recommendations, and any other information considered as part of predicted patient outcome in a remote database, such as in the patient's EMR as described above in step 518.

### Heart Failure Deployment

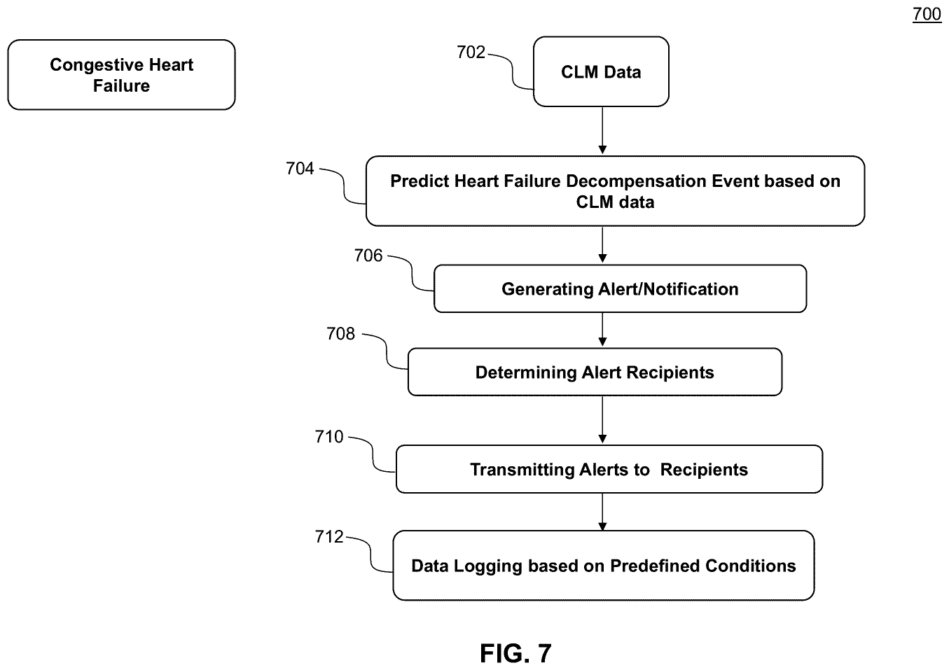

FIG. 7 is a flowchart illustrating a method 700 for utilizing a continuous analyte monitoring system in the detection of heart failure conditions, according to some embodiments. Method 700 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 7 may be performed by a components of CAM system 100, either separately or in a distributed fashion. In such an embodiment, any of these components may execute code in memory to perform certain steps of method 700 of FIG. 7. While method 700 of FIG. 7 will be discussed below as being performed by certain components of CAM system 100, other components may store the code and therefore may execute method 700 by directly executing the code. Accordingly, the following discussion of method 700 will refer to components of FIGS. 1-3 as an exemplary non-limiting embodiment. Moreover, it is to be appreciated that not all steps may be needed to perform the disclosure provided herein. Further, some of the functions may be performed simultaneously, in a different order, or by the same components than shown in FIG. 7, as will be understood by a person of ordinary skill in the art.

Heart failure (HF) is a condition where the cardiac output is inadequate to meet the demands of the body. HF presents with symptoms of shortness of breath, particularly when lying flat, swelling of the legs and ankles, and reduced ability to exercise. Patients with heart failure vary in severity from those who develop symptoms on exertion whereas the more severe patients have symptoms at rest. HF is often a chronic condition, where the patient experiences a slow, decline in cardiac function. However, some patients can experience a sudden deterioration in cardiac function, known as acute heart failure, and frequently requires adjustment of treatment and often needing hospitalization. During episodes of heart failure, the heart pumps inadequately and cannot pump blood out to the body normally. Blood backs up in the lungs and increases blood pressure there. Inability of the heart to relax appropriately can also cause blood to back up into the lungs, which contributes to pulmonary hypertension. When the heart is not able to pump efficiently, blood can back up into the veins that take blood through the lungs. As the pressure in these blood vessels increases, fluid is pushed into the air spaces (alveoli) in the lungs. Typically, an implantable pulmonary artery pressure sensor, such as the CardioMEMS HF system is utilized to monitor rising pulmonary artery pressure, which indicates right ventricular congestion (afterload). Monitoring lactate levels through an on-body sensor is non-invasive and can provide information for predicting changes to pulmonary artery pressure. This is possible due to the CAM system 100 utilizing rising lactate as an indicator for a potential heart failure decompensation event.

Accordingly, a prediction model in CAM system 100 may rely on continuous lactate monitoring to monitor blood lactate levels for the purpose of detecting and predicting cardiac events in patients. The prediction model may utilize changes in lactate levels to provide an early and non-invasive means for predicting rising pulmonary artery pressure which can lead to earlier treatment and monitoring. For example, pulmonary artery predictions from the prediction model can prompt adjustment to treatment by one or more clinicians. As previously discussed, the prediction model may be trained using outcome (e.g., health records, patient data, cohort data, prior predictions, etc.) and lactate levels. When trained, current lactate information (e.g., provided by sensor control device 102) may be input to the prediction model for generating a predicted patient outcome. In some embodiments, other inputs may be provided to the prediction model as well including, but not limited to, patient data and patient vitals.

In HF, the inefficient pumping of the heart leads to a build-up of pressure within the heart. This build-up of pressure is typically measured in the pulmonary artery. The prediction model of CAM system 100 receives lactate information in a patient and may be configured to generate predictions related to a heart failure based on correlating the lactate information with a rise (or decline) in pulmonary pressure. That is, the prediction model may generate a prediction of increased HF risk based on an increase in the lactate levels of the patient.

In some embodiments, in order to distinguish between lactate level increases due to exercise and lactate level increases due to potential heart failure decompensation events, analyte sensor control device 102 may be configured to include or communicate with an accelerometer and/or altimeter so as to monitor patient activity during a lactate excursion. Patient activity may be used as a factor for predicting heart failure decompensation events, for example, by minimizing significance of lactate level increase during increased levels of patient activity (e.g., which could indicate exercise or physical activity as causing the increase in lactate levels).

In some aspects, the prediction model utilizes cumulative sum lactate to form its prediction for heart failure decompensation events. Cumulative sum lactate is a running total of lactate over a predetermined period of time (e.g., 8 hours, 24 hours, 2 days) and may be used to illustrate the total amount of lactate as it rises over that predetermined period of time.

In 702, a prediction model of CAM system 100 receives continuously monitored lactate information from one or more patients (e.g., via analyte sensor control device 102 connected to each respective patient). As previously noted, the prediction model may be implemented within one or more components in CAM system 100, such as any combination of data receiving device 120, multi-purpose data receiving device 130, and/or user device 135.

In 704, the prediction model may then utilize the CLM data for predicting potential heart failure decompensation events for the patient. The CLM data may be provided in the form of a cumulative sum of lactate levels over a predetermined period of time. This predetermined period of time may be based on any number of factors include HCP preference, desired level of accuracy for the prediction, and determined severity of patient's condition.

In 706, a component of CAM system 100 (e.g., any combination of data receiving device 120, multi-purpose data receiving device 130, and/or user device 135) may generate an alert and/or notification based on any predicted heart failure decompensation events in the patient. Alerts and notifications may include the predicted patient heart failure decompensation event. The alert may further include one or more treatment recommendations for addressing the predicted patient heart failure decompensation event. A component of CAM system 100 may generate the treatment recommendations based on predicted patient heart failure decompensation event and include the generated recommendations in the alert. Accordingly, an alert and/or notification may be generated to notify one or more recipients of the patient's predicted patient heart failure decompensation event, and an alert may further may provide recommendation actions for addressing the predicted event.

In 708, a component in CAM system 100 (e.g., any combination of data receiving device 120, multi-purpose data receiving device 130, and/or user device 135) may determine the recipients for receiving the generated alert and/or notification. The recipients may be predetermined based on different predicted patient heart failure decompensation events and proximity to patient, time of day, and level of severity of the predicted patient heart failure decompensation events. When implemented in a home setting, the intended recipient may be identified based on their capability to more quickly respond to any alerts that require immediate action.

In 710, the alert and/or notification is transmitted to a device(s) associated with the predetermined recipient(s). Examples of devices associated with include their personal devices (with dedicated software installed) for receiving the alert and/or notification.

In 712, remote application server 155 may receive and store the patient information, including the predicted patient heart failure decompensation events, the recommendations, and any other data associated with the patient, in a remote database, such as in the patient's EMR as described above in step 518.

### Sepsis Deployment

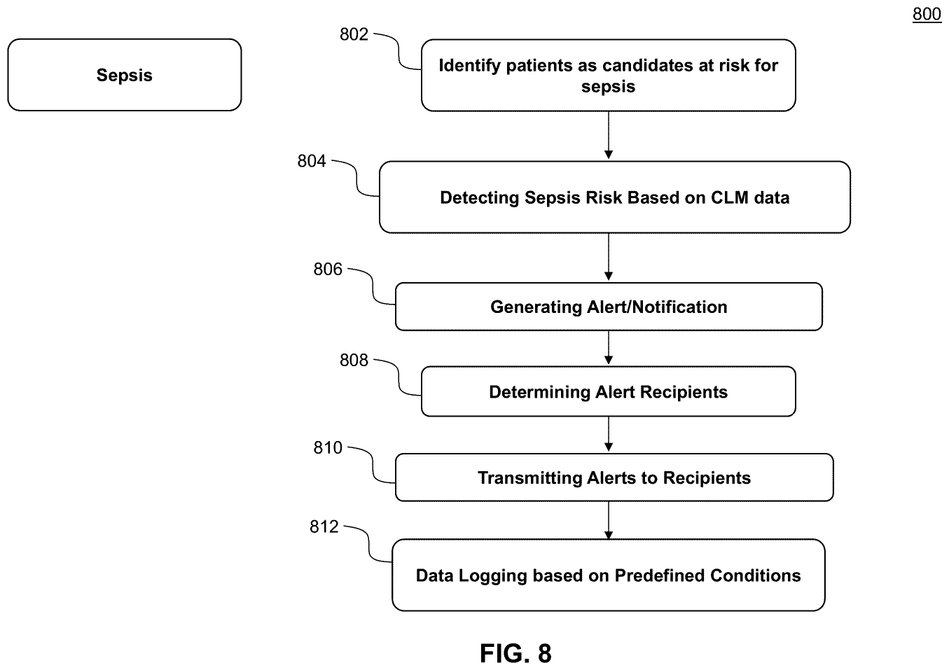

FIG. 8 is a flowchart illustrating a method 800 for utilizing CAM system 100 for the detection of sepsis conditions, according to some embodiments. Method 800 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 8 may be performed by a components of CAM system 100, either separately or in a distributed fashion. In such an embodiment, any of these components may execute code in memory to perform certain steps of method 800 of FIG. 8. While method 800 of FIG. 8 will be discussed below as being performed by certain components of CAM system 100, other components may store the code and therefore may execute method 800 by directly executing the code. Accordingly, the following discussion of method 800 will refer to components of FIGS. 1-3 as an exemplary non-limiting embodiment. Moreover, it is to be appreciated that not all steps may be needed to perform the disclosure provided herein. Further, some of the functions may be performed simultaneously, in a different order, or by the same components than shown in FIG. 8, as will be understood by a person of ordinary skill in the art. Sepsis is the body's extreme response to an infection. It is a life-threatening medical emergency. Sepsis happens when an infection triggers an exaggerated reaction from the body's immune system, causing significant morbidity and mortality. Some people are at higher risk of developing sepsis because they are at higher risk of contracting an infection. These include the very young (infants), the very old, those with chronic illnesses, and those with a weakened or impaired immune system.

CAM system 100 is configured to measure lactate levels in dermal interstitial fluid allowing for closer monitoring of the lactate levels of patients suspected of having sepsis in the hospital and home settings. In particular, continuous lactate monitoring provides a novel means of measuring lactate levels for predicting the likelihood of sepsis in patients. CAM system 100 provides an increased level of surveillance of a patient's lactate levels with the potential of reducing the requirement for multiple blood tests, allowing for dynamic adjustments to treatment and therapy based on predicted likelihood of sepsis. A prediction model in CAM system 100 is configured to correlate rises in lactate levels with certain outcomes, including sepsis, and the prediction provided by the prediction model may be used to direct treatment. For example, during acute illnesses such as sepsis, levels of the hormone epinephrine are raised which leads to the conversion of glucose into lactate, increasing circulating the level of lactate in the patient. In addition, poor tissue oxygenation, primarily due to a drop in blood pressure, leads to anaerobic respiration which further contributes to lactate levels. The prediction model utilizes a patient's lactate information (e.g., columns 1006 from trend table 1000), including current levels and trending data along with data specific to the patient (e.g., trend data from column 1002 from trend table 1000) in order to generate a sepsis prediction.

Consequently, CAM system 100, when configured for continuous lactate measurement, may be used to predict the deterioration of a patient at risk of developing sepsis, monitor a patient's response to treatment of the sepsis, and provide a means of surveillance in patients recovering from sepsis who are at risk of recurrence, and predict additional clinical outcome based on the sepsis information and monitored information.

In 802, a component of CAM system 100 (e.g., any combination of data receiving device 120, multi-purpose data receiving device 130, and/or user device 135) receives patient identification of any patients that are identified candidates for continuous remote monitoring based on a potential risk for sepsis. For example, during discharge from a hospital or during a visit to the doctor's visit, an HCP may determine that the patient is at risk for sepsis that would merit continuous remote monitoring including the continuous remote monitoring of a patient's analyte levels. Once identified, a patient identifier associated with the patient may be CAM system 100 to indicate that the analyte sensor control device 102 associated with the patient is authorized to communicate with components of the CAM system 100 and should be used for predicting potential sepsis risk in the patient. In some embodiments, the prediction model relies on continuous lactate data (as opposed to a single measurement such as from a blood draw) which provides serial lactate measurements of a patient over a predetermined of time. Predictions of sepsis conditions can be more accurate when utilizing continuous lactate data.

In 804, the prediction model may then utilize the CLM data for predicting potential sepsis conditions for the patient. The CLM data may be provided in the form of a cumulative sum of lactate levels over a predetermined period of time. The predetermined period of time be based on any number of factors include HCP preference, desired level of accuracy for the prediction, and determined severity of patient's condition. The prediction may also be based on prior treatment and the prediction model is providing a means of surveillance in patients recovering from sepsis and may be at risk of recurrence.

In 806, a component of CAM system 100 (e.g., any combination of data receiving device 120, multi-purpose data receiving device 130, and/or user device 135) may generate an alert and/or notification based on the detection of a predicted sepsis condition. Alerts and notifications include the predicted sepsis condition. The alert may include one or more treatment recommendations for addressing the predicted sepsis condition. A component of CAM system 100 may generate the treatment recommendations based on the predicted patient outcome generated in 506 and include the generated recommendations in the alert. Accordingly, an alert and/or notification may be generated to notify one or more recipients of the patient's predicted sepsis condition.

In 808, a component in CAM system 100 (e.g., any combination of data receiving device 120, multi-purpose data receiving device 130, and/or user device 135) may determine the recipients for receiving the generated alert and/or notification. The recipients may be predetermined based on different predicted sepsis condition, proximity to patient, time of day, and level of severity of the predicted sepsis condition. When implemented in a home setting, the intended recipient may be identified based on their capability to more quickly respond to any alerts that require immediate action.

In 810, the alert and/or notification is transmitted to a device associated with the predetermined recipient. Examples of devices associated with include their personal devices (with dedicated software installed) for receiving the alert and/or notification.

In 812, remote application server 155 may receive and store the patient information including the predicted sepsis, the recommendations, and any other data associated with the patient, in a remote database, such as in the patient's EMR as described above in step 518.

### High Risk Surgery Deployment

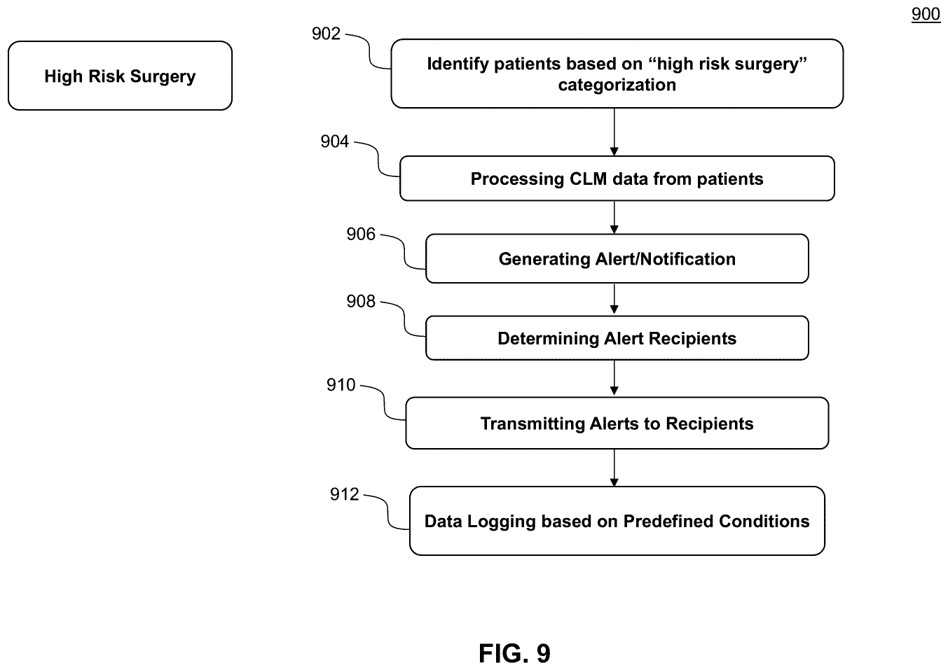

FIG. 9 is a flowchart illustrating a method 900 for utilizing a continuous analyte monitoring system during high-risk surgery procedures, according to some embodiments. Method 900 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 9 may be performed by a components of CAM system 100, either separately or in a distributed fashion. In such an embodiment, any of these components may execute code in memory to perform certain steps of method 900 of FIG. 9. While method 900 of FIG. 9 will be discussed below as being performed by certain components of CAM system 100, other components may store the code and therefore may execute method 900 by directly executing the code. Accordingly, the following discussion of method 900 will refer to components of FIGS. 1-3 as an exemplary non-limiting embodiment. Moreover, it is to be appreciated that not all steps may be needed to perform the disclosure provided herein. Further, some of the functions may be performed simultaneously, in a different order, or by the same components than shown in FIG. 9, as will be understood by a person of ordinary skill in the art.

No surgery is without risk; however, some procedures have a higher risk of complication than others. Examples of surgical conditions which carry a high risk of developing perioperative complications, such as sepsis and hypovolemia, include, orthopaedic surgery, gastrointestinal surgery, vascular surgery, obstetric surgery, bariatric surgery, surgery in elderly patients, surgery in patients with comorbidities such as diabetes, heart failure, etc., and generally any surgery with prolonged anaesthesia.

Continuous lactate monitoring by CAM system 100 provides a means for real-time measurement of lactate levels in high-risk surgical settings. Rises in lactate levels are associated with worse clinical outcomes and continuous lactate monitoring could be used to detect patient decline earlier and to direct treatment decisions. A prediction model in CAM system 100 is configured to correlate rises in lactate levels with certain outcomes in high-risk surgical settings and the prediction provided by the prediction model may be used to anticipate potential patient conditions and dynamically adapt treatment of patients during their recovery period.

In 902, CAM system 100 receives identification of any patients that have undergone surgical procedures categorized as “high-risk.” For example, before or after a high-risk surgical procedure, an HCP may identify the patient undergoing the procedure as a candidate for continuous monitoring of analyte levels by CAM system 100. Once identified, a patient identifier associated with the patient may be CAM system 100 to indicate that the analyte sensor control device 102 associated with the patient is authorized to communicate with components, including the prediction model, of the CAM system 100.

In 904, the prediction model may then utilize the CLM data for predicting potential deterioration conditions for the patient following the high-risk surgical procedure. The CLM data may be provided in the form of a cumulative sum of lactate levels over a predetermined period of time. The predetermined period of time be based on any number of factors include HCP preference, desired level of accuracy for the prediction, and determined severity of patient's condition. The prediction may also be based on the type of high-risk surgical procedure, any treatment occurring before and after the high-risk surgical procedure, and any other medical information associated with the patient including current vital signs. The prediction model is providing a means of surveillance in patients recovering from high-risk surgery and may be at risk of complications during recovery.

In 906, a component of CAM system 100 (e.g., any combination of data receiving device 120, multi-purpose data receiving device 130, and/or user device 135) may generate an alert and/or notification based on the detection of any signs of deterioration in the patient. Alerts and notifications include the predicted deterioration conditions. The alert may include one or more treatment recommendations for addressing the predicted deterioration conditions. A component of CAM system 100 may generate the treatment recommendations based on the predicted deterioration conditions generated in 506 and include the generated recommendations in the alert. Accordingly, an alert and/or notification may be generated to notify one or more recipients of the patient's predicted deterioration conditions.

In 908, a component in CAM system 100 (e.g., any combination of data receiving device 120, multi-purpose data receiving device 130, and/or user device 135) may determine the recipients for receiving the generated alert and/or notification. The recipients may be predetermined based on different predicted deterioration conditions, proximity to patient, time of day, and level of severity of the predicted deterioration conditions. When implemented in a home setting (e.g., when the patient is recovering at home after the high-risk surgical procedure), the intended recipient may be identified based on their capability to more quickly respond to any alerts that require immediate action.

In 910, the alert and/or notification is transmitted to a device associated with the predetermined recipient. Examples of devices associated with include their personal devices (with dedicated software installed) for receiving the alert and/or notification.

In 912, remote application server 155 may receive and store the patient information, including the predicted patient deterioration conditions, the recommendations, and any other data associated with the patient, in a remote database, such as in the patient's EMR as described above in step 518.

The present disclosure is further illustrated by the following embodiments.
(1) A computer implemented method for predicting patient outcome based on continuous analyte data, the method comprising:
   receiving, by a processor implemented in an early warning system, the continuous analyte data, wherein the continuous analyte data is associated with a patient, and wherein the continuous analyte data is provided by a continuous analyte sensor associated with the patient;
   generating, by the processor, a predicted patient outcome based on the continuous analyte data;
   identifying, by the processor and based on the predicted patient outcome and the continuous analyte data, a predetermined recipient device; and
   transmitting, by the processor, a notification to the predetermined recipient device, wherein the notification includes the predicted patient outcome.
(2) The method of (1), wherein the processor is in communication with a patient prediction model, and wherein the patient prediction model is a machine learning model.
(3) The method of (2), wherein the predicted patient outcome is generated by the patient prediction model, wherein the method further comprises:
   inputting, to the patient prediction model, the continuous analyte data; and
   outputting, by the patient prediction model to the processor, the predicted patient outcome.
(4) The method of (1, 2 or 3), wherein the notification further includes a recommendation for treating the predicted patient outcome.
(5) The method of any of (1) to (4), wherein the predicted patient outcome is further based on patient medical information, and wherein the method further comprises: inputting, to the patient prediction model, the patient medical information in combination with the continuous analyte data, wherein the patient medical information includes one or more of patient procedure history, patient medical history, or current vital signs associated with the patient.
(6) The method of any one of (1) to (5), further comprising retrieving, by the processor, a use-case deployment associated with the patient, and wherein the use-case deployment includes one of a hospital setting, a home setting, a sepsis condition, a heart failure condition, or a high-risk surgery condition.
(7) The method of any one of (1) to (6), wherein the analyte sensor is a dual-analyte sensor.
(8) The method of (7), wherein the first and second continuous analyte data comprise any combination of two analytes from lactate, glucose, creatinine, and ketone.
(9) The method of any of (1) to (8), further comprising determining a trend in a value of the analyte and an absolute value of the analyte based on the continuous analyte data, and generating the predicted patient outcome based on both the determined trend and absolute value of the analyte.
(10) The method of any of (1) to (9), further comprising determining a trend in a value of the analyte and an absolute value of the analyte based on the continuous analyte data, and determining the content of the notification and/or recipient of the notification based on both the determined trend and absolute value of the analyte.
(11) The method of (9) or (10), wherein:
   when the determined absolute value of the analyte is below a first threshold and the determined trend is above a second threshold, the method comprises sending an alert to a device associated with a determined recipient; and
   when the determined absolute value of the analyte is below the first threshold and the determined trend is below the second threshold, no alert is sent.
(12) The method of (11), wherein:
   when the determined absolute value of the analyte is below the first threshold, the determined trend is above the second threshold, and the use-case deployment is a home setting, the method comprises sending an alert to a device associated with a determined recipient;
   when the determined absolute value of the analyte is below the first threshold, the determined trend is above the second threshold but below a third threshold higher than the second threshold, and the use-case deployment is a hospital setting, no alert is sent; and when the determined absolute value of the analyte is below the first threshold, the determined trend is above the third threshold, and the use-case deployment is a hospital setting, the method comprises sending an alert to a device associated with a determined recipient.
(13) The method of (11) or (12), wherein:
   when the determined absolute value of the analyte is above a fourth threshold and the determined trend is above a fifth threshold, the method comprises sending an alert to a device associated with a determined recipient; and
   when the determined absolute value of the analyte is above the fourth threshold and the determined trend is below the fifth threshold, no alert is sent.
(14) The method of any one of (8) to (13), wherein the trend is a rate of change of the value of the analyte over a given time period, wherein the given time period comprises fifteen minutes, one hour, two hours, or three or more hours.
(15) The method of (6), further comprising determining the content of the notification and/or recipient of the notification based on the predicted patient outcome and/or determined use-case deployment.
(16) The method of (15), wherein the content of the notification and/or recipient of the notification is determined based on one or more of the proximity of the recipient to the patient, time of day, and level of severity of the predicted patient outcome.
(17) The method of (15) or (16), wherein the notification includes an instruction to administer an intervention to the patient based on the predicted patient outcome, and the recipient of the notification is a medical device configured to automatically administer the intervention in response to the instruction.
(18) The method of (17), wherein the intervention is one or more of fluid resuscitation, administration of antibiotics, or provision of breathing oxygen gas.
(19) The method of any preceding clause, wherein the notification includes an instruction for adjusting or maintaining a dosage of a substance to be administered to the patient, and the recipient of the notification is a device configured to administer the substance to the patient based on the dosage specified in the instruction.
(20) The method of (19), wherein the treatment device is an insulin injection pen or insulin pump and the analyte is glucose.
(21) The method of any preceding clause, wherein notifications are sent at a frequency based on the use-case deployment.
(22) The method of any one of (11) to (14), wherein the continuous analyte data is received at a frequency based on the determined trend, the frequency configured to increase when the determined trend is above a another threshold.
(23) The method of any preceding clauses, wherein the notification comprises an alert notifying a caregiver and/or the patient that an immediate intervention is required.
(24) The method of any of (1) to (23), wherein the predicted patient outcome comprises a determination of a particular disease, optionally heart failure or sepsis.
(25) A computer-implemented method for operating an early warning system based on continuous analyte data, the method comprising:
   receiving, by a processor implemented in the early warning system, first continuous analyte data and second continuous analyte data, wherein the first continuous analyte data is associated with a first patient and the second continuous analyte data is associated with a second patient, and wherein the first continuous analyte data is provided by a first continuous analyte sensor associated with the first patient and the second continuous analyte data is provided by a second continuous analyte sensor associated with the second patient;
   determining, by the processor in communication with the first continuous analyte sensor and the second continuous analyte sensor, a first use-case deployment based on at least one of a first user preference and a monitored condition of the first patient and a second use-case deployment based on at least one a second user preference and a monitored condition of the second patient;
   providing, by the processor, the first use-case deployment and the first continuous analyte data as a first input to a patient prediction model;
   providing, by the processor, the second use-case deployment and the second continuous analyte data as a second input to the patient prediction model, wherein the first use-case deployment differs from the second use-case deployment;
   receiving, from the patient prediction model, a first predicted patient outcome associated with the first patient and a second predicted patient outcome associated with the second patient;
   identifying, based on the first predicted patient outcome, a first predetermined recipient device, and based on the second predicted patient, a second predetermined recipient device; and
   transmitting a first notification, generated based on the first predicted patient outcome, to the first predetermined remote device, and a second notification, generated based on the second predicted patient outcome, to the second predetermined remote device.
(26) The method of (25), wherein the patient prediction model is a machine learning model.
(27) The method of (25 or 26), wherein the notification further includes a recommendation for treating the predicted patient outcome.
(28) The method of (25) to (27), wherein the predicted patient outcome is generated by a patient prediction model, wherein the method further comprises: inputting, to the patient prediction model, the continuous analyte data; and outputting, by the patient prediction model, the predicted patient outcome.
(29) The method of any one of (25) to (28), wherein the predicted patient outcome is further based on patient medical information, and wherein the method further comprises:
   inputting, to the patient prediction model, the patient medical information in combination with the continuous analyte data, wherein the patient medical information includes one or more of patient procedure history, patient medical history, or current vital signs associated with the patient.
(30) The method of any one of (25) to (29), wherein the use-case deployment includes one of a hospital setting, a home setting, a sepsis condition, a heart failure condition, or a high-risk surgery condition.
(31) The method of any one of (25) to (30), wherein the analyte sensor is a dual-analyte sensor and the first and second continuous analyte data comprise any combination of two analytes from lactate, glucose, creatinine, and ketone.
(32) The method of any of (25) to (31), further comprising determining a trend in a value of the analyte and an absolute value of the analyte based on the continuous analyte data, and generating the predicted patient outcome based on both the determined trend and absolute value of the analyte.
(33) The method of any of (25) to (32), further comprising determining a trend in a value of the analyte and an absolute value of the analyte based on the continuous analyte data, and determining the content of the notification and/or recipient of the notification based on both the determined trend and absolute value of the analyte.
(34) The method of (32) or (33), wherein:
   when the determined absolute value of the analyte is below a first threshold and the determined trend is above a second threshold, the method comprises sending an alert to a device associated with a determined recipient; and
   when the determined absolute value of the analyte is below the first threshold and the determined trend is below the second threshold, no notification is transmitted.
(35) The method of (34), wherein:
   when the determined absolute value of the analyte is below the first threshold, the determined trend is above the second threshold, and the use-case deployment is a home setting, the method comprises sending an alert to a device associated with a determined recipient;
   when the determined absolute value of the analyte is below the first threshold, the determined trend is above the second threshold but below a third threshold higher than the second threshold, and the use-case deployment is a hospital setting, no alert is sent; and
   when the determined absolute value of the analyte is below the first threshold, the determined trend is above the third threshold, and the use-case deployment is a hospital setting, the method comprises sending an alert to a device associated with a determined recipient.
(36) The method of (34) or (35), wherein:
   when the determined absolute value of the analyte is above a fourth threshold and the determined trend is above a fifth threshold, the method comprises sending an alert to a device associated with a determined recipient; and
   when the determined absolute value of the analyte is above the fourth threshold and the determined trend is below the fifth threshold, no alert is sent.
(37) The method of any one of (32) to (36), wherein the trend is a rate of change of the value of the analyte over a given time period, wherein the given time period comprises fifteen minutes, one hour, two hours, or three or more hours.
(38) The method of any preceding clause, further comprising determining the content of the notification and/or recipient of the notification based on the predicted patient outcome and/or determined use-case deployment.
(39) The method of (38), wherein the content of the notification and/or recipient of the notification is determined based on one or more of the proximity of the recipient to the patient, time of day, and level of severity of the predicted patient outcome.
(40) The method of (38) or (39), wherein the notification includes an instruction to administer an intervention to the patient based on the predicted patient outcome, and the recipient of the notification is a medical device connected to the patient and configured to automatically administer the intervention in response to the instruction.
(41) The method of (40), wherein the intervention is one or more of fluid resuscitation, administration of antibiotics, or provision of breathing oxygen gas.
(42) The method of any preceding clause, wherein the notification includes an instruction for adjusting or maintaining a dosage of a substance to be administered to the patient, and the recipient of the notification is a device configured to administer the substance to the patient based at the dosage specified in the instruction.
(43) The method of (42), wherein the treatment device is an insulin injection pen or insulin pump and the analyte is glucose.
(44) The method of any preceding clause, wherein notifications are sent at a frequency based on the use-case deployment.
(45) The method of any one of (42) to (44), wherein the continuous analyte data is received at a frequency based on the determined trend, the frequency configured to increase when the determined trend is above a given threshold.
(46) The method of any preceding clause, wherein the notification comprises an alert notifying a caregiver and/or the patient that an immediate intervention is required.
(47) An early warning system configured to perform the method of any preceding clause.
(48) An early warning system configured to perform the method of any preceding clause, wherein the prediction model is implemented on any of one combination of a data receiving device, multi-purpose data receiving device, and/or user device.
(49) An early warning system configured to perform the method of any preceding clause, wherein the prediction model comprises at least one of first prediction model configured for a hospital deployment, a second prediction model configured for a home deployment, a third prediction model configured for a heart failure deployment, a fourth prediction model configured for a sepsis deployment, and a fifth prediction model configured for a high-risk surgery deployment.

### CONCLUSION

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A method for operating an early warning system based on continuous analyte data, the method comprising:
receiving, by a processor implemented in the early warning system, continuous analyte data, wherein the continuous analyte data is associated with a patient, and wherein the continuous analyte data is provided by a continuous analyte sensor associated with the patient;
determining, by the processor in communication with the continuous analyte sensor, a use-case deployment based on at least one of a user preference and a monitored condition of the patient;
determining a trend in the continuous analyte data and an absolute value of an analyte value based on the continuous analyte data;
providing, by the processor, the use-case deployment, the determined trend, the absolute value of an analyte value, and the continuous analyte data as inputs to a patient prediction model;
receiving, from the patient prediction model, a predicted patient outcome associated with the first patient;
identifying a predetermined recipient device based on the predicted patient outcome; and
transmitting a notification, generated based on the first predicted patient outcome, to the predetermined recipient device.

2. The method of claim 1, wherein the notification comprises a recommendation for treating the first predicted patient outcome.

3. The method of claim 1, wherein the predicted patient outcome is generated by the patient prediction model, wherein the method further comprises:
inputting, to the patient prediction model, the continuous analyte data; and
outputting, by the patient prediction model, the predicted patient outcome.

4. The method of claim 1, wherein the patient prediction model is a machine learning model.

5. The method of claim 1, wherein the predicted patient outcome is further based on patient medical information, and wherein the method further comprises:
inputting, to the patient prediction model, patient medical information in combination with the continuous analyte data, wherein the patient medical information includes one or more of patient procedure history, patient medical history, or current vital signs associated with the first patient.

6. The method of claim 1, wherein the use-case deployment includes one of a hospital setting, a home setting, a sepsis condition, a heart failure condition, or a high-risk surgery condition.

7. The method of claim 1, wherein the continuous analyte sensor is a dual-analyte sensor and the continuous analyte data comprises lactate and any combination of glucose, creatinine, and ketone.

8. The method of claim 1, wherein the notification includes an instruction for adjusting or maintaining a dosage of a substance to be administered to the patient, and the predetermined recipient device is configured to administer the substance to the patient based at the dosage specified in the instruction.

9. The method of claim 1, wherein content of the notification is determined based on one or more of a proximity of a recipient of the predetermined recipient device to the patient, a time of day, and level of severity of the predicted patient outcome and identifying the predetermined recipient device is further based on one or more of the proximity of the recipient to the patient, the time of day, and the level of severity of the predicted patient outcome.

10. The method of claim 1, further comprising:
receiving, by the processor, second continuous analyte data, wherein the second continuous analyte data is associated with a second patient, and wherein the second continuous analyte data is provided by a second continuous analyte sensor associated with the second patient;
determining, by the processor in communication with the second continuous analyte sensor, a second use-case deployment based on at least one a second user preference and a monitored condition of the second patient;
providing, by the processor, the second use-case deployment and the second continuous analyte data as a second input to the patient prediction model, wherein the use-case deployment differs from the second use-case deployment;
receiving, from the patient prediction model, a second predicted patient outcome associated with the second patient;
identifying a second predetermined recipient device based on the second predicted patient outcome; and
transmitting a second notification, generated based on the second predicted patient outcome, to the second predetermined recipient device.

11. The method of claim 1, wherein:
when the determined absolute value of an analyte value in the continuous analyte data is below a first threshold and the determined trend is above a second threshold, the method comprises sending, by the processor, an alert to the predetermined recipient device; and
when the determined absolute value of the analyte value is below the first threshold and the determined trend is below the second threshold, preventing transmission of the alert.

12. The method of claim 11, wherein:
when the determined absolute value of the analyte value is below the first threshold, the determined trend is above the second threshold, and the use-case deployment is a home setting, the method comprises sending a second alert to a device associated with another predetermined recipient device;
when the determined absolute value of the analyte value is below the first threshold, the determined trend is above the second threshold but below a third threshold higher than the second threshold, and the use-case deployment is a hospital setting, preventing transmission of the second alert; and
when the determined absolute value of the analyte value is below the first threshold, the determined trend is above the third threshold, and the use-case deployment is the hospital setting, the method comprises sending an alert to a device associated with a different predetermined recipient device.

13. The method of claim 11, wherein the determined trend is a rate of change of the analyte value over a given time period.

14. The method of claim 1, further comprising determining content of the notification based on the predicted patient outcome and the use-case deployment.

15. The method of claim 1, wherein a content of the notification is determined based on one or more of a proximity of a recipient associated with the predetermined recipient device to the patient, time of day, and a level of severity of the predicted patient outcome.

16. The method of claim 15, wherein the content of the notification includes an instruction to administer an intervention to the patient based on the predicted patient outcome, and the predetermined recipient device is configured to automatically administer the intervention in response to the instruction.

17. The method of claim 16, wherein the intervention is one or more of fluid resuscitation, administration of antibiotics, provision of breathing oxygen gas.

18. The method of claim 16, wherein the notification includes an instruction for adjusting or maintaining a dosage of a substance to be administered, and the predetermined recipient device is configured to administer the substance to the patient based at the dosage specified in the instruction.

19. An early warning system, comprising:
a first continuous analyte sensor configured to continuously collect first continuous analyte data of a first patient and a second continuous analyte sensor configured to continuously collect second continuous analyte data of a second patient;
one or more processors in communication with the first continuous analyte sensor and the second continuous analyte sensor; and
a memory coupled to the one or more processors and storing a prediction model and instructions that when executed by the one or more processors cause the one or more processors to:
receive continuous analyte data, wherein the continuous analyte data is associated with a patient, and wherein the continuous analyte data is provided by a continuous analyte sensor associated with the patient;
determine a use-case deployment based on at least one of a user preference and a monitored condition of the first patient;
determine a trend in the continuous analyte data and an absolute value of an analyte value based on the continuous analyte data;
provide the use-case deployment, the determined trend, the absolute value of an analyte value, and the continuous analyte data as inputs to a patient prediction model;
receive, from the patient prediction model, a predicted patient outcome associated with the first patient;
identify a predetermined recipient device based on the predicted patient outcome; and
transmit a notification, generated based on the first predicted patient outcome, to the predetermined recipient device.

20. A non-transitory, tangible computer-readable medium having instructions stored thereon that, when executed by at least one computing device in an early warning system, cause a processor of the at least one computing device to perform operations comprising:
receiving, by the processor, continuous analyte data, wherein the continuous analyte data is associated with a patient, and wherein the continuous analyte data is provided by a continuous analyte sensor associated with the patient;
determining, by the processor in communication with the continuous analyte sensor, a use-case deployment based on at least one of a user preference and a monitored condition of the patient;
determining a trend in the continuous analyte data and an absolute value of an analyte value based on the continuous analyte data;
providing, by the processor, the use-case deployment, the determined trend, the absolute value of an analyte value, and the continuous analyte data as inputs to a patient prediction model;
receiving, from the patient prediction model, a predicted patient outcome associated with the first patient;
identifying a predetermined recipient device based on the predicted patient outcome; and
transmitting a notification, generated based on the first predicted patient outcome, to the predetermined recipient device.
